(19) United States
(12) Patent Application Publication
(10) Pub. No.: US 2023/0250195 A1
(43) Pub. Date: Aug. 10, 2023

(54) Methods for Reducing Infusion-Related Reactions in Patients Treated with EGFR/Met Bispecific Antibodies

(71) Applicant: Janssen Biotech, Inc., Horsham, PA (US)

(72) Inventors: Trishala Agrawal, Horsham, PA (US); Parthiv Mahadevia, Raritan, NJ (US)

(21) Appl. No.: 18/106,531

(22) Filed: Feb. 7, 2023

Related U.S. Application Data
(60) Provisional application No. 63/307,375, filed on Feb. 7, 2022.
(60) Provisional application No. 63/389,042, filed on Jul. 14, 2022.

(51) Int. Cl. C07K 16/46; A61K 31/573; A61K 31/47; A61K 31/519; A61K 31/5377; A61K 31/506; A61K 31/135; A61P 37/06

(52) U.S. Cl. CPC C07K 16/468; A61K 31/573; A61K 31/47; A61K 31/519; A61K 31/5377; A61K 31/506; A61K 31/135; A61P 37/06

(57) ABSTRACT

The present invention relates to methods of reducing occurrence or severity of infusion-related reactions (IRRs) in a subject treated with an anti-epidermal growth factor receptor (EGFR)/hepatocyte growth factor receptor (c-Met) antibody, comprising administering (a) dexamethasone; (b) montelukast; or (c) methotrexate to the subject.

### CROSS-REFERENCE TO RELATED APPLICATIONS

This application claims the benefit of U.S. Provisional Patent Application No. 63/307,375, filed Feb. 7, 2022, and U.S. Provisional Patent Application No. 63/389,042, filed Jul. 14, 2022, the disclosure of which is herein incorporated by reference in its entirety.

### REFERENCE TO SEQUENCE LISTING SUBMITTED ELECTRONICALLY

The sequence listing of the present application is submitted electronically via The United States Patent and Trademark Center Patent Center as an XML formatted sequence listing with a file name “JBI6706USNP1SEQLIST.xml”, creation date of Jan. 31, 2023, and a size of 20 kilobytes (KB). This sequence listing submitted is part of the specification and is herein incorporated by reference in its entirety.

### FIELD OF THE INVENTION

The present invention relates to methods of reducing occurrence or severity of infusion-related reactions (IRRs) in a subject treated with an anti-epidermal growth factor receptor (EGFR)/hepatocyte growth factor receptor (c-Met) antibody, comprising administering (a) dexamethasone; (b) montelukast; or (c) methotrexate to the subject.

### BACKGROUND

Amivantamab is a bispecific EGF receptor-directed and MET receptor-directed antibody FDA approved for the treatment of adult patients with locally advanced or metastatic NSCLC with EGFR exon 20 insertion mutations, as detected by an FDA-approved test, whose disease has progressed on or after platinum-based chemotherapy. Amivantamab administration can cause infusion-related reactions (IRRs) in a proportion of patients. Signs and symptoms of IRR include but are not limited to dyspnea, flushing, fever, chills, nausea, chest discomfort, hypotension, and vomiting. Systemic IRRs, including severe reactions, upon the introduction of a new protein therapeutic infusion are frequently observed but the mechanisms inducing the reactions are varied.

### SUMMARY

There is a need for improved combinations of therapeutics that would reduce the infusion-related reactions (IRRs) in patients treated with EGFR/c-Met bispecific antibodies, such as amivantamab.

The disclosure generally relates to methods that are useful for treating IRRs in patients treated with EGFR/c-Met bispecific antibodies.

In one aspect, the disclosure provides a method of reducing occurrence or severity of infusion-related reactions (IRRs) in a subject treated with an anti-epidermal growth factor receptor (EGFR)/hepatocyte growth factor receptor (c-Met) antibody, comprising administering (a) dexamethasone; (b) montelukast; or (c) methotrexate.

In some embodiments, the antibody comprises:
a first domain that specifically binds EGFR, comprising heavy chain complementarity determining region 1 (HCDR1), HCDR2, HCDR3, light chain complementarity determining region 1 (LCDR1), LCDR2 and LCDR3 amino acid sequences of SEQ ID NOs: 1, 2, 3, 4, 5 and 6, respectively; and
a second domain that specifically binds c-Met, comprising HCDR1, HCDR2, HCDR3, LCDR1, LCDR2 and LCDR3 amino acid sequences of SEQ ID NOs: 7, 8, 9, 10, 11 and 12, respectively.

In some embodiments, the first domain comprises a heavy chain variable region (VH) of SEQ ID NO:13 and a light chain variable region (VL) of SEQ ID NO:14, and the second domain comprises a VH of SEQ ID NO:15 and a VL of SEQ ID NO:16.

In some embodiments, the antibody is of the IgG1 isotype.

In some embodiments, the antibody comprises a first heavy chain (HC1) of SEQ ID NO:17, a first light chain (LC1) of SEQ ID NO:18, a second heavy chain (HC2) of SEQ ID NO:19 and a second light chain (LC2) of SEQ ID NO:20.

In some embodiments, the antibody is an isolated bispecific antibody.

In some embodiments, the bispecific antibody is amivantamab.

In some embodiments, the antibody is administered at a dose of about 700 mg to about 1,400 mg.

In some embodiments, the antibody is administered at a dose of about 700 mg, about 1,050 mg or about 1,400 mg.

In some embodiments, the antibody is administered at a dose of about 1,400 mg.

In some embodiments, the antibody is administered at a dose of about 1,050 mg.

In some embodiments, the antibody is administered at a dose of about 700 mg.

In some embodiments, the antibody is administered once a week or once every two weeks.

In some embodiments, the antibody is administered once weekly for the first 4 weeks and then every 2 weeks.

In some embodiments, the antibody is administered as a monotherapy.

In some embodiments, the subject treated with the anti-EGFR/c-Met antibody is further administered one or more chemotherapeutic agents.

In some embodiments, the one or more chemotherapeutic agents comprise a tyrosine kinase inhibitor (TKI).

In some embodiments, the one or more chemotherapeutic agents comprise Lazertinib.

In some embodiments, the one or more chemotherapeutic agents comprise osimertinib.

In some embodiments, methotrexate is administered between 7 days to 3 days prior to the administration of the anti-EGFR/c-Met antibody.

In some embodiments, methotrexate is administered at a dose of 25 mg.

In some embodiments, montelukast is administered daily starting 4 days prior to the administration of the anti-EGFR/c-Met antibody.

In some embodiments, montelukast is administered 5 times.

In some embodiments, montelukast is administered at a dose 10 mg.

In some embodiments, the method further comprises administration of IV dexamethasone on the first and second days of administering the anti-EGFR/c-Met antibody, wherein the administration of dexamethasone is 45-60 minutes prior to the administration of the anti-EGFR/c-Met antibody.

In some embodiments, IV dexamethasone is administered at a dose of 10 mg.

In some embodiments, oral dexamethasone is administered 1 day prior to the administration of the anti-EGFR/c-Met antibody.

In some embodiments, oral dexamethasone is administered at a total daily dose of 8 mg.

In some embodiments, the method further comprises administration of IV dexamethasone on the first and second days of administering the anti-EGFR/c-Met antibody, wherein IV dexamethasone is administered at a dose between 10-20 mg.

In some embodiments, the method further comprises administering a premedication with one or more of antihistamines, antipyretics, or glucocorticoids.

In some embodiments, the premedication further comprises diphenhydramine.

In some embodiments, the diphenhydramine is administered at a dose of 25 to 50 mg.

In some embodiments, the premedication further comprises acetaminophen.

In some embodiments, the acetaminophen is administered at a dose of 650 to 1,000 mg.

### DETAILED DESCRIPTION

### Definitions

All publications, including but not limited to patents and patent applications, cited in this specification are herein incorporated by reference as though fully set forth.

It is to be understood that the terminology used herein is for describing particular embodiments only and is not intended to be limiting. Unless defined otherwise, all technical and scientific terms used herein have the same meaning as commonly understood by one of ordinary skill in the art to which the invention pertains.

Although any methods and materials similar or equivalent to those described herein may be used in the practice for testing of the present invention, exemplary materials and methods are described herein. In describing and claiming the present invention, the following terminology will be used.

When a list is presented, unless stated otherwise, it is to be understood that each individual element of that list, and every combination of that list, is a separate embodiment. For example, a list of embodiments presented as “A, B, or C” is to be interpreted as including the embodiments, “A,” “B,” “C,” “A or B,” “A or C,” “B or C,” or “A, B, or C.”

As used in this specification and the appended claims, the singular forms “a,” “an,” and “the” include plural referents unless the content clearly dictates otherwise. Thus, for example, reference to “a cell” includes a combination of two or more cells, and the like.

The conjunctive term “and/or” between multiple recited elements is understood as encompassing both individual and combined options. For instance, where two elements are conjoined by “and/or,” a first option refers to the applicability of the first element without the second. A second option refers to the applicability of the second element without the first. A third option refers to the applicability of the first and second elements together. Any one of these options is understood to fall within the meaning, and therefore satisfy the requirement of the term “and/or” as used herein. Concurrent applicability of more than one of the options is also understood to fall within the meaning, and therefore satisfy the requirement of the term “and/or.”

The transitional terms “comprising,” “consisting essentially of,” and “consisting of” are intended to connote their generally accepted meanings in the patent vernacular; that is, (i) “comprising,” which is synonymous with “including,” “containing,” or “characterized by,” is inclusive or open-ended and does not exclude additional, unrecited elements or method steps; (ii) “consisting of” excludes any element, step, or ingredient not specified in the claim; and (iii) “consisting essentially of” limits the scope of a claim to the specified materials or steps “and those that do not materially affect the basic and novel characteristic(s)” of the claimed invention. Embodiments described in terms of the phrase “comprising” (or its equivalents) also provide as embodiments those independently described in terms of “consisting of” and “consisting essentially of.”

“About” means within an acceptable error range for the particular value as determined by one of ordinary skill in the art, which will depend in part on how the value is measured or determined, i.e., the limitations of the measurement system. Unless explicitly stated otherwise within the Examples or elsewhere in the Specification in the context of a particular assay, result or embodiment, “about” means within one standard deviation per the practice in the art, or a range of up to 5%, whichever is larger.

The term “antibody” or “antibodies” is meant in a broad sense and includes immunoglobulin molecules including monoclonal antibodies including murine, human, humanized and chimeric monoclonal antibodies, full-length antibodies, antigen binding fragments, multispecific antibodies, such as bispecific, trispecific, tetraspecific etc., dimeric, tetrameric or multimeric antibodies, single chain antibodies, domain antibodies and any other modified configuration of the immunoglobulin molecule that comprises an antigen binding site of the required specificity.

“Specific binding” or “specifically binds” or “specifically binding” or “binds” refer to an antibody binding to an antigen or an epitope within the antigen with greater affinity than for other antigens. Typically, the antibody binds to the antigen or the epitope within the antigen with an equilibrium dissociation constant (K_{D}) of about 5×10⁻⁸ M or less, for example about 1×10⁻⁹ M or less, about 1×10⁻¹⁰ M or less, about 1×10⁻¹¹ M or less, or about 1×10⁻¹² M or less, typically with the K_{D} that is at least one hundred-fold less than its K_{D} for binding to a non-specific antigen (e.g., BSA, casein). The dissociation constant may be measured using known protocols. Antibodies that bind to the antigen or the epitope within the antigen may, however, have cross-reactivity to other related antigens, for example to the same antigen from other species (homologs), such as human or monkey, for example Macaca fascicularis (cynomolgus, cyno) or Pan troglodytes (chimpanzee, chimp). While a monospecific antibody binds one antigen or one epitope, a bispecific antibody binds two distinct antigens or two distinct epitopes.

“Complementarity determining regions” (CDR) are antibody regions that bind an antigen. CDRs may be defined using various delineations such as Kabat (Wu et al. (1970) J Exp Med 132: 211-50) (Kabat et al., Sequences of Proteins of Immunological Interest, 5th Ed. Public Health Service, National Institutes of Health, Bethesda, Md., 1991), Chothia (Chothia et al. (1987) J Mol Biol 196: 901-17), IMGT (Lefranc et al. (2003) Dev Comp Immunol 27: 55-77) and AbM (Martin and Thornton (1996) J Bmol Biol 263: 800-15). The correspondence between the various delineations and variable region numbering are described (see e.g., Lefranc et al. (2003) Dev Comp Immunol 27: 55-77; Honegger and Pluckthun, (2001) J Mol Biol 309:657-70; International ImMunoGeneTics (IMGT) database; Web resources, http://www_imgt_org). Available programs such as abYsis by UCL Business PLC may be used to delineate CDRs. The term “CDR”, “HCDR1”, “HCDR2”, “HCDR3”, “LCDR1”, “LCDR2” and “LCDR3” as used herein includes CDRs defined by any of the methods described supra, Kabat, Chothia, IMGT or AbM, unless otherwise explicitly stated in the specification.

“Full-length antibodies” are comprised of two heavy chains (HC) and two light chains (LC) inter-connected by disulfide bonds as well as multimers thereof (e.g., IgM). Each heavy chain is comprised of a heavy chain variable region (VH) and a heavy chain constant region (comprised of domains CH1, hinge, CH2 and CH3). Each light chain is comprised of a light chain variable region (VL) and a light chain constant region (CL). The VH and the VL regions may be further subdivided into regions of hypervariability, termed complementarity determining regions (CDR), interspersed with framework regions (FR). Each VH and VL is composed of three CDRs and four FR segments, arranged from amino-to-carboxy-terminus in the following order: FR1, CDR1, FR2, CDR2, FR3, CDR3 and FR4.

“Antigen binding fragment” refers to a portion of an immunoglobulin molecule that binds an antigen. Antigen binding fragments may be synthetic, enzymatically obtainable or genetically engineered polypeptides and include the VH, the VL, the VH and the VL, Fab, F(ab′)2, Fd and Fv fragments, domain antibodies (dAb) consisting of one VH domain or one VL domain, shark variable IgNAR domains, camelized VH domains, minimal recognition units consisting of the amino acid residues that mimic the CDRs of an antibody, such as FR3-CDR3-FR4 portions, the HCDR1, the HCDR2 and/or the HCDR3 and the LCDR1, the LCDR2 and/or the LCDR3. VH and VL domains may be linked together via a synthetic linker to form various types of single chain antibody designs where the VH/VL domains may pair intramolecularly, or intermolecularly in those cases when the VH and VL domains are expressed by separate single chain antibody constructs, to form a monovalent antigen binding site, such as single chain Fv (scFv) or diabody; described for example in Int. Patent Publ. Nos. WO1998/44001, WO1988/01649, WO1994/13804 and WO1992/01047.

“Monoclonal antibody” refers to an antibody obtained from a substantially homogenous population of antibody molecules, i.e., the individual antibodies comprising the population are identical except for possible well-known alterations such as removal of C-terminal lysine from the antibody heavy chain or post-translational modifications such as amino acid isomerization or deamidation, methionine oxidation or asparagine or glutamine deamidation. Monoclonal antibodies typically bind one antigenic epitope. A bispecific monoclonal antibody binds two distinct antigenic epitopes. Monoclonal antibodies may have heterogeneous glycosylation within the antibody population. Monoclonal antibody may be monospecific or multispecific such as bispecific, monovalent, bivalent or multivalent.

“Humanized antibodies” refers to antibodies in which the antigen binding sites are derived from non-human species and the variable region frameworks are derived from human immunoglobulin sequences. Humanized antibodies may include intentionally introduced mutations in the framework regions so that the framework may not be an exact copy of expressed human immunoglobulin or germline gene sequences.

“Human antibodies” refers to antibodies having heavy and light chain variable regions in which both the framework and the antigen binding site are derived from sequences of human origin. If the antibody contains a constant region or a portion of the constant region, the constant region is also derived from sequences of human origin. Antibodies in which antigen binding sites are derived from a non-human species are not included in the definition of “human antibody.”

A human antibody comprises heavy or light chain variable regions that are derived from sequences of human origin if the variable regions of the antibody are obtained from a system that uses human germline immunoglobulin or rearranged immunoglobulin genes. Non-limiting example systems include human immunoglobulin gene libraries displayed on phage, and transgenic non-human animals such as mice or rats carrying human immunoglobulin loci. A human antibody typically contains amino acid differences when compared to the human germline or rearranged immunoglobulin sequences due to, for example, naturally occurring somatic mutations, intentional substitutions in the framework or antigen binding site, and substitutions introduced during cloning or VDJ recombination in non-human animals. Typically, a human antibody is at least 80% identical in amino acid sequence to an amino acid sequence encoded by a human germline or rearranged immunoglobulin gene. For example, about: 80%, 81%, 82%, 83%, 84%, 85%, 86%, 87%, 88%, 89%, 90%, 91%, 92%, 93%, 94%, 95%, 96%, 97%, 98%, 99% or 100% identical. In some cases, a human antibody may contain consensus framework sequences derived from human framework sequence analyses (see, e.g., Knappik et al., J. Mol. Biol. 296:57-86 (2000)), or synthetic HCDR3 incorporated into human immune-globulin gene libraries displayed on phage (see, e.g., Shi et al., J. Mol. Biol. 397:385-96 (2010) and Int. Pat. Publ. No. WO2009/085462).

“Bispecific” refers to an antibody that specifically binds two distinct antigens or two distinct epitopes within the same antigen. The bispecific antibody may have cross-reactivity to other related antigens, for example to the same antigen from other species (homologs), such as human or monkey, for example Macaca cynomolgus (cynomolgus, cyno) or Pan troglodytes, or may bind an epitope that is shared between two or more distinct antigens.

“Bispecific anti-EGFR/c-Met antibody” or “bispecific EGFR/c-Met antibody” refers to a bispecific antibody having a first domain that specifically binds EGFR and a second domain that specifically binds c-Met. The domains specifically binding EGFR and c-Met are typically VH/VL pairs, and the bispecific anti-EGFR/c-Met antibody is monovalent in terms of binding to EGFR and c-Met.

“Isolated” refers to a homogenous population of molecules (such as synthetic polynucleotides, polypeptides vectors or viruses) which have been substantially separated and/or purified away from other components of the system the molecules are produced in, such as a recombinant cell, as well as a protein that has been subjected to at least one purification or isolation step. “Isolated” refers to a molecule that is substantially free of other cellular material and/or chemicals and encompasses molecules that are isolated to a higher purity, such as to 80%, 81%, 82%, 83%, 84%, 85%, 86%, 87%, 88%, 89%, 90%, 91%, 92%, 93%, 94%, 95%, 96%, 97%, 98%, 99% or 100% purity.

Immunoglobulins may be assigned to five major classes, IgA, IgD, IgE, IgG and IgM, depending on the heavy chain constant domain amino acid sequence. IgA and IgG are further sub-classified as the isotypes IgA1, IgA2, IgG1, IgG2, IgG3 and IgG4. Antibody light chains of any vertebrate species may be assigned to one of two clearly distinct types, namely kappa (κ) and lambda (λ), based on the amino acid sequences of their constant domains.

“Low fucose” or “low fucose content” as used in the application refers to antibodies with fucose content of about between 1%-15%.

“Normal fucose” or “normal fucose content” as used herein refers to antibodies with fucose content of about over 50%, typically about over 80% or over 85%.

“Recombinant” refers to DNA, antibodies and other proteins that are prepared, expressed, created or isolated by recombinant means when segments from different sources are joined to produce recombinant DNA, antibodies or proteins.

“Carrier” refers to a diluent, adjuvant, excipient, or vehicle with which the antibody of the invention is administered. Such vehicles may be liquids, such as water and oils, including those of petroleum, animal, vegetable or synthetic origin, such as peanut oil, soybean oil, mineral oil, sesame oil and the like. For example, 0.4% saline and 0.3% glycine may be used to formulate the bispecific anti-EGFR/c-Met antibody. These solutions are sterile and generally free of particulate matter. They may be sterilized by conventional, well-known sterilization techniques (e.g., filtration). For parenteral administration, the carrier may comprise sterile water and other excipients may be added to increase solubility or preservation. Injectable suspensions or solutions may also be prepared utilizing aqueous carriers along with appropriate additives. Suitable vehicles and formulations, inclusive of other human proteins, e.g., human serum albumin, are described, for example, in e.g., Remington: The Science and Practice of Pharmacy, 21^{st} Edition, Troy, D. B. ed., Lippincott Williams and Wilkins, Philadelphia, Pa. 2006, Part 5, Pharmaceutical Manufacturing pp 691-1092, See especially pp. 958-989.

“Dosage” refers to the information of the amount of the therapeutic or the drug to be taken by the subject and the frequency of the number of times the therapeutic is to be taken by the subject. “Dose” refers to the amount or quantity of the therapeutic or the drug to be taken each time.

“Therapeutically effective amount” refers to an amount effective, at doses and for periods of time necessary, to achieve a desired therapeutic result. A therapeutically effective amount may vary depending on factors such as the disease state, age, sex, and weight of the individual, and the ability of a therapeutic or a combination of therapeutics to elicit a desired response in the individual. Exemplary indicators of an effective therapeutic or combination of therapeutics that include, for example, improved well-being of the patient.

“Co-administration,” “administration with,” “administration in combination with,” “in combination with” or the like, encompass administration of the selected therapeutics or drugs to a single patient, and are intended to include treatment regimens in which the therapeutics or drugs are administered by the same or different route of administration or at the same or different time.

“Fixed combination” refers to a single pharmaceutical composition comprising two or more compounds.

“Non-fixed combination” refers to separate pharmaceutical compositions, wherein each comprises one or more compounds. The one or more compounds or unit dosage forms can be administered as separate entities either simultaneously, concurrently or sequentially with no specific intervening time limits, wherein such administration provides effective levels of the two compounds in the body of the subject.

“Antagonist” or “inhibitor” refers to a molecule that, when bound to a cellular protein, suppresses at least one reaction or activity that is induced by a natural ligand of the protein. A molecule is an antagonist when the at least one reaction or activity is suppressed by at least about 20%, 30%, 40%, 45%, 50%, 55%, 60%, 65%, 70%, 75%, 80%, 85%, 90%, 95%, or 100% more than the at least one reaction or activity suppressed in the absence of the antagonist (e.g., negative control), or when the suppression is statistically significant when compared to the suppression in the absence of the antagonist.

“Treat”, “treating” or “treatment” of a disease or disorder such as cancer refers to accomplishing one or more of the following: reducing the severity and/or duration of the disorder, inhibiting worsening of symptoms characteristic of the disorder being treated, limiting or preventing recurrence of the disorder in subjects that have previously had the disorder, or limiting or preventing recurrence of symptoms in subjects that were previously symptomatic for the disorder.

“Prevent”, “preventing”, “prevention”, or “prophylaxis” of a disease or disorder means preventing that a disorder occurs in subject.

“Responsive”, “responsiveness” or “likely to respond” refers to any kind of improvement or positive response, such as alleviation or amelioration of one or more symptoms, diminishment of extent of disease, stabilized (i.e., not worsening) state of disease, preventing spread of disease, delay or slowing of disease progression, amelioration or palliation of the disease state, and remission (whether partial or total), whether detectable or undetectable.

“Subject” includes any human or nonhuman animal. “Nonhuman animal” includes all vertebrates, e.g., mammals and non-mammals, such as nonhuman primates, sheep, dogs, cats, horses, cows, chickens, amphibians, reptiles, etc. The terms “subject” and “patient” are used interchangeably herein.

“Cancer” refers to an abnormal growth of cells which tend to proliferate in an uncontrolled way and, in some cases, to metastasize (spread) to other areas of a patient's body.

“EGFR or c-Met expressing cancer” refers to cancer that has detectable expression of EGFR or c-Met or has EGFR or c-Met mutation or amplification. EGFR or c-Met expression, amplification and mutation status can be detected using know methods, such as sequencing, next generation sequencing, fluorescent in situ hybridization, immunohistochemistry, flow cytometry or western blotting.

“Epidermal growth factor receptor” or “EGFR” refers to the human EGFR (also known as HER1 or ErbB1 (Ullrich et al., Nature 309:418-425, 1984) having the amino acid sequence shown in GenBank accession number NP_005219, as well as naturally-occurring variants thereof.

“Hepatocyte growth factor receptor” or “c-Met” as used herein refers to the human c-Met having the amino acid sequence shown in GenBank Accession No: NP_001120972 and natural variants thereof.

“Newly diagnosed” refers to a subject who has been diagnosed with EGFR or c-Met expressing cancer but has not yet received treatment for CRC (e.g., mCRC).

“Refractory” refers to a disease that does not respond to a treatment. A refractory disease can be resistant to a treatment before or at the beginning of the treatment, or a refractory disease can become resistant during a treatment.

“Relapsed” refers to the return of a disease or the signs and symptoms of a disease after a period of improvement after prior treatment with a therapeutic.

“Diagnosing” or “diagnosis” refers to methods to determine if a subject is suffering from a given disease or condition or may develop a given disease or condition in the future or is likely to respond to treatment for a prior diagnosed disease or condition, i.e., stratifying a patient population on likelihood to respond to treatment. Diagnosis is typically performed by a physician based on the general guidelines for the disease to be diagnosed or other criteria that indicate a subject is likely to respond to a particular treatment.

“Biological sample” refers to a collection of similar fluids, cells, or tissues isolated from a subject, as well as fluids, cells, or tissues present within a subject. Exemplary samples are biological fluids such as blood, serum and serosal fluids, plasma, lymph, urine, saliva, cystic fluid, tear drops, feces, sputum, mucosal secretions of the secretory tissues and organs, vaginal secretions, ascites fluids, fluids of the pleural, pericardial, peritoneal, abdominal and other body cavities, fluids collected by bronchial lavage, synovial fluid, liquid solutions contacted with a subject or biological source, for example, cell and organ culture medium including cell or organ conditioned medium, lavage fluids and the like, tissue biopsies, tumor tissue biopsies, tumor tissue samples, fine needle aspirations, surgically resected tissue, organ cultures or cell cultures.

### Methods of the Disclosure

In one aspect, the disclosure provides a method of reducing occurrence or severity of infusion-related reactions (IRRs) in a subject treated with an anti-epidermal growth factor receptor (EGFR)/hepatocyte growth factor receptor (c-Met) antibody, comprising administering one or more of methotrexate, montelukast, or dexamethasone.

In one aspect, the disclosure provides a method of reducing occurrence or severity of infusion-related reactions (IRRs) in a subject treated with a combination treatment, comprising an anti-epidermal growth factor receptor (EGFR)/hepatocyte growth factor receptor (c-Met) antibody, comprising administering one or more of methotrexate, montelukast, or dexamethasone.

### Methods for IRR Reduction

The disclosure provides a method of reducing occurrence or severity of infusion-related reactions (IRR) in a subject who is treated with an antibody that specifically binds an anti-epidermal growth factor receptor (EGFR) and/or hepatocyte growth factor receptor (c-Met). In one embodiment, the subject is treated with a combination treatment, comprising an antibody that specifically binds EGFR and/or c-Met. In one embodiment, the subject is treated with a combination treatment, comprising amivantamab.

The EGFR/c-Met bispecific antibody administered parenterally can cause an adverse reaction or adverse event (AE) in a patient or subject, specifically an infusion-related reaction (IRR). As reported in the United States Prescribing Information for RYBREVANT (amivantamab) (www.janssenlabels.com/package-insert/product-monograph/prescribing-information/RYBREVANT-pi.pdf), among 302 patients with locally advanced or metastatic non-small cell lung cancer (NSCLC) who received IV amivantamab at recommended Phase 2 dose (RP2D) as a single agent in the CHRYSALIS study, IRRs were among the most commonly occurring adverse reactions with an incidence of 66%.

An adverse reaction or adverse event (AE) is any untoward medical occurrence in a patient or a subject to whom EGFR/c-Met bispecific antibody is being administered or was administered. In some embodiments, the AE is an IRR. In some embodiments, the IRRs are mild IRR, manifesting as, but not limited to, chills, nausea, dyspnea, flushing, chest discomfort, hypotension, vomiting, tachycardia, fever, or any other symptoms during the time of the infusion or after the infusion. In some embodiments, the IRRs are systemic IRRs, including severe reactions, upon the introduction of a new protein therapeutic infusion, such as EGFR/c-Met bispecific antibody. In some embodiments, the EGFR/c-Met bispecific antibody is amivantamab. In some embodiments, the severity of IRRs is graded according to the National Cancer Institute Common Terminology Criteria for Adverse Events (NCI-CTCAE) version 5.0. Severity scale ranges from Grade 1 (Mild) to Grade 5 (Death). Grade 1=Mild, Grade 2=Moderate, Grade 3=Severe, Grade 4=Life-threatening and Grade 5=Death related to adverse event. In some embodiments, the presence of IRRs in a patient or subject is evaluated at any time starting at Cycle 1 Day 1 and ending 30 days after end of treatment with the EGFR/c-Met bispecific antibody. In some embodiments, the presence of IRRs in a patient or subject is evaluated at Cycle 1 Day 1 of treatment with the EGFR/c-Met bispecific antibody. In some embodiments, the presence of IRRs in a patient or subject is evaluated at a time up to 3 month after the start of treatment with the EGFR/c-Met bispecific antibody. In some embodiments, the presence of IRRs in a patient or subject is evaluated at any time between 1 day to 30 days after end of treatment with the EGFR/c-Met bispecific antibody. In some embodiments, the presence of IRRs in a patient or subject is evaluated at any time between 1 day to 5 days after end of treatment with the EGFR/c-Met bispecific antibody. In some embodiments, the presence of IRRs in a patient or subject is evaluated at any time between 1 day to 10 days after end of treatment with the EGFR/c-Met bispecific antibody. In some embodiments, the presence of IRRs in a patient or subject is evaluated at any time between 1 day to 15 days after end of treatment with the EGFR/c-Met bispecific antibody. In some embodiments, the presence of IRRs in a patient or subject is evaluated at any time between 1 day to 20 days after end of treatment with the EGFR/c-Met bispecific antibody.

In some embodiments, the method of reducing occurrence or severity of IRRs comprises administering one or more of methotrexate, montelukast, or dexamethasone. In some embodiments, the method of reducing occurrence or severity of IRRs comprises administering methotrexate. In some embodiments, the method of reducing occurrence or severity of IRRs comprises administering montelukast. In some embodiments, the method of reducing occurrence or severity of IRRs comprises administering dexamethasone.

### Methotrexate

Methotrexate (MTX) [N-[4-[[(2,4-diamino-6-pteridinyl)methyl]methylamino]benzoyl]-Lglutamic acid], is an FDA-approved folic acid antagonist indicated for the treatment of rheumatoid arthritis. MTX is an anti-metabolite, commonly used in chemotherapy and immunosuppressant in auto-immune diseases. Methotrexate inhibits dihydrofolic acid reductase, interfering with DNA synthesis, repair, and cellular replication. Actively proliferating tissues such as malignant cells, bone marrow, fetal cells, buccal and intestinal mucosa, and cells of the urinary bladder are in general more sensitive to this effect of methotrexate.

In some embodiments, methotrexate is administered subcutaneously (SC). In some embodiments, methotrexate is administered orally. In some embodiments, methotrexate is administered intramuscularly. In some embodiments, methotrexate is administered intravenously.

In some embodiments, methotrexate is administered several days prior to the treatment with EGFR/c-Met bispecific antibody. In some embodiments, methotrexate is administered 1-7 days prior to the treatment with EGFR/c-Met bispecific antibody. In some embodiments, methotrexate is administered 1-5 days prior to the treatment with EGFR/c-Met bispecific antibody. In some embodiments, methotrexate is administered 1-6 days prior to the treatment with EGFR/c-Met bispecific antibody. In some embodiments, methotrexate is administered 1-4 days prior to the treatment with EGFR/c-Met bispecific antibody. In some embodiments, methotrexate is administered 1-3 days prior to the treatment with EGFR/c-Met bispecific antibody. In some embodiments, methotrexate is administered 1-2 days prior to the treatment with EGFR/c-Met bispecific antibody. In some embodiments, methotrexate is administered 3-7 days prior to the treatment with EGFR/c-Met bispecific antibody. In some embodiments, methotrexate is administered 3-6 days prior to the treatment with EGFR/c-Met bispecific antibody. In some embodiments, methotrexate is administered 3-5 days prior to the treatment with EGFR/c-Met bispecific antibody. In some embodiments, methotrexate is administered 2-7 days prior to the treatment with EGFR/c-Met bispecific antibody.

In some embodiments, methotrexate is administered 1 day prior to the treatment with EGFR/c-Met bispecific antibody. In some embodiments, methotrexate is administered 2 days prior to the treatment with EGFR/c-Met bispecific antibody. In some embodiments, methotrexate is administered 3 days prior to the treatment with EGFR/c-Met bispecific antibody. In some embodiments, methotrexate is administered 4 days prior to the treatment with EGFR/c-Met bispecific antibody. In some embodiments, methotrexate is administered 5 days prior to the treatment with EGFR/c-Met bispecific antibody. In some embodiments, methotrexate is administered 6 days prior to the treatment with EGFR/c-Met bispecific antibody. In some embodiments, methotrexate is administered 7 days prior to the treatment with EGFR/c-Met bispecific antibody. In some embodiments, methotrexate is administered 8 days prior to the treatment with EGFR/c-Met bispecific antibody.

In some embodiments, methotrexate is administered in a dose of 12-30 mg. In some embodiments, methotrexate is administered in a total dose of 20-30 mg. In some embodiments, methotrexate is administered in a total dose of 25 mg. In some embodiments, methotrexate is administered in a single dose of 20-30 mg. In some embodiments, methotrexate is administered in a single dose of 25 mg. In some embodiments, methotrexate is administered in a single dose of 20 mg. In some embodiments, methotrexate is administered in a single dose of 21 mg. In some embodiments, methotrexate is administered in a single dose of 22 mg. In some embodiments, methotrexate is administered in a single dose of 23 mg. In some embodiments, methotrexate is administered in a single dose of 24 mg. In some embodiments, methotrexate is administered in a single dose of 26 mg. In some embodiments, methotrexate is administered in a single dose of 27 mg. In some embodiments, methotrexate is administered in a single dose of 28 mg. In some embodiments, methotrexate is administered in a single dose of 29 mg. In some embodiments, methotrexate is administered in a single dose of 30 mg.

In some embodiments, methotrexate is administered as a single dose. In some embodiments, methotrexate is administered as two doses. In some embodiments, methotrexate is administered as two or more doses.

### Montelukast

Montelukast is an oral medication, FDA-approved for the treatment of chronic asthma and prophylaxis and the prevention of exercise-induced bronchoconstriction. It is also approved for the relief of symptoms of both seasonal and perennial allergic rhinitis. Montelukast inhibits the mast cell mediated release of leukotrienes and may be used to reduce inflammation and bronchoconstriction. Montelukast is a highly selective leukotriene receptor antagonist that binds with high affinity to leukotrienes, which are excreted by various types of cells, including mast cells, and are involved in the inflammatory process that may cause the signs and symptoms of asthma and allergic rhinitis. Leukotriene receptors are found in airway cells, such as macrophages and smooth muscle cells. When bound to leukotriene receptors, montelukast inhibits leukotriene physiologic effects (such as airway edema, smooth muscle contraction, and impairment of normal cellular activity). This serves as the rationale for montelukast to potentially reduce the symptomatology (e.g. dyspnea) associated with EGFR/c-Met bispecific antibody IRRs.

In some embodiments, montelukast is administered orally. In some embodiments, montelukast is administered as an injection. In some embodiments, montelukast is administered subcutaneously. In some embodiments, montelukast is administered intravenously. In some embodiments, montelukast is administered intramuscularly.

In some embodiments, montelukast is administered prior to the treatment with EGFR/c-Met bispecific antibody. In some embodiments, montelukast is administered 1-7 times prior to the treatment with EGFR/c-Met bispecific antibody. In some embodiments, montelukast is administered 1 time prior to the treatment with EGFR/c-Met bispecific antibody. In some embodiments, montelukast is administered 2 times prior to the treatment with EGFR/c-Met bispecific antibody. In some embodiments, montelukast is administered 3 times prior to the treatment with EGFR/c-Met bispecific antibody. In some embodiments, montelukast is administered 4 times prior to the treatment with EGFR/c-Met bispecific antibody. In some embodiments, montelukast is administered 5 times prior to the treatment with EGFR/c-Met bispecific antibody. In some embodiments, montelukast is administered 6 times prior to the treatment with EGFR/c-Met bispecific antibody. In some embodiments, montelukast is administered 7 times prior to the treatment with EGFR/c-Met bispecific antibody.

In some embodiments, montelukast is administered starting 5 days prior to the treatment with EGFR/c-Met bispecific antibody. In some embodiments, montelukast is administered starting 4 days prior to the treatment with EGFR/c-Met bispecific antibody. In some embodiments, montelukast is administered starting 3 days prior to the treatment with EGFR/c-Met bispecific antibody. In some embodiments, montelukast is administered starting 2 days prior to the treatment with EGFR/c-Met bispecific antibody. In some embodiments, montelukast is administered starting 2 days prior to the treatment with EGFR/c-Met bispecific antibody. In some embodiments, montelukast is administered starting 4 days prior to the treatment with EGFR/c-Met bispecific antibody, wherein the last administration of montelukast is given on the first day of treatment with the EGFR/c-Met bispecific antibody.

In some embodiments, montelukast is administered at a dose of 4-20 mg. In some embodiments, montelukast is administered at a dose of 5-10 mg. In some embodiments, montelukast is administered at a dose of 4 mg. In some embodiments, montelukast is administered at a dose of 5 mg. In some embodiments, montelukast is administered at a dose of 8 mg. In some embodiments, montelukast is administered at a dose of 10 mg. In some embodiments, montelukast is administered at a dose of 15 mg. In some embodiments, montelukast is administered at a dose of 16 mg. In some embodiments, montelukast is administered at a dose of 20 mg.

### Dexamethasone

Dexamethasone is a synthetic adrenocortical steroid available in oral and IV formulations FDA-approved for allergic states. IV dexamethasone (10 mg) is a standard premedication administered prior to all patients receiving IV amivantamab. The disclosure provides a novel method of enhanced steroid pre-loading to reduce the incidence of EGFR/c-Met bispecific antibody IRRs.

In some embodiments, dexamethasone is administered orally. In some embodiments, dexamethasone is administered subcutaneously. In some embodiments, dexamethasone is administered intravenously. In some embodiments, dexamethasone is administered intramuscularly.

In some embodiments, dexamethasone is administered prior to the treatment with EGFR/c-Met bispecific antibody. In some embodiments, dexamethasone is administered 2 days prior to the treatment with EGFR/c-Met bispecific antibody. In some embodiments, dexamethasone is administered 1 day prior to the treatment with EGFR/c-Met bispecific antibody. In some embodiments, dexamethasone is administered 3 or more days prior to the treatment with EGFR/c-Met bispecific antibody.

In some embodiments, dexamethasone is administered once per day. In some embodiments, dexamethasone is administered twice per day.

In some embodiments, dexamethasone is administered at a dose 2-10 mg. In some embodiments, dexamethasone is administered at a dose 4-8 mg. In some embodiments, dexamethasone is administered at a dose of 4 mg. In some embodiments, dexamethasone is administered at a dose of 8 mg.

In some embodiments, IV dexamethasone is administered in addition to methotrexate, montelukast, or oral dexamethasone.

In some embodiments, IV dexamethasone is administered at a dose of 10-20 mg. In some embodiments, IV dexamethasone is administered at a dose of 10 mg. In some embodiments, IV dexamethasone is administered at a dose of 15 mg. In some embodiments, IV dexamethasone is administered at a dose of 20 mg.

In some embodiments, IV dexamethasone is administered 45-60 minutes prior to IV EGFR/c-Met bispecific antibody. In some embodiments, IV dexamethasone is administered on the same day as IV EGFR/c-Met bispecific antibody, prior to IV EGFR/c-Met bispecific antibody. In some embodiments, IV dexamethasone is administered on the first day and on the second day of IV EGFR/c-Met bispecific antibody administration.

### Patient Population

In some embodiments, the subject who is treated with an antibody that specifically binds EGFR and/or c-Met, has been diagnosed with cancer. In some embodiments, the cancer is a solid tumor. In some embodiments, the solid tumor is lung cancer, a non-small cell lung cancer or a small cell lung cancer. In some embodiments, the cancer is a non-small cell lung cancer (NSCLC). In some embodiments, the solid tumor is colorectal cancer (CRC). In some embodiments, the solid tumor is liver cancer. In some embodiments, the solid tumor is hepatocellular carcinoma. In some embodiments, the solid tumor is gastric cancer. In some embodiments, the solid tumor is esophageal cancer. In some embodiments, the solid tumor is head and neck cancer. In some embodiments, the solid tumor is squamous cell carcinoma of the head and neck.

In some embodiments, the cancer is the epithelial cell cancer. In some embodiments, the cancer is the breast cancer. In some embodiments, the cancer is the ovarian cancer. In some embodiments, the cancer is the lung cancer. In some embodiments, the cancer is the lung adenocarcinoma. In some embodiments, the cancer is the squamous cell lung cancer. In some embodiments, the cancer is the small cell lung cancer. In some embodiments, the cancer is the colorectal cancer. In some embodiments, the cancer is the anal cancer. In some embodiments, the cancer is the prostate cancer. In some embodiments, the cancer is the kidney cancer. In some embodiments, the cancer is the bladder cancer. In some embodiments, the cancer is the head and neck cancer. In some embodiments, the cancer is the pharynx cancer. In some embodiments, the cancer is the cancer of the nose. In some embodiments, the cancer is the pancreatic cancer. In some embodiments, the cancer is the skin cancer. In some embodiments, the cancer is the oral cancer. In some embodiments, the cancer is the cancer of the tongue. In some embodiments, the cancer is the esophageal cancer. In some embodiments, the cancer is the vaginal cancer. In some embodiments, the cancer is the cervical cancer. In some embodiments, the cancer is the cancer of the spleen. In some embodiments, the cancer is the testicular cancer. In some embodiments, the cancer is the gastric cancer. In some embodiments, the cancer is the cancer of the thymus. In some embodiments, the cancer is the colon cancer. In some embodiments, the cancer is the thyroid cancer. In some embodiments, the cancer is the liver cancer. In some embodiments, the cancer is the HCC. In some embodiments, the cancer is the PRCC.

In some embodiments, the cancer is EGFR or c-Met expressing cancer.

In some embodiments, the cancer is EGFR and c-Met expressing cancer.

In some embodiments, the cancer is EGFR expressing cancer.

In some embodiments, the cancer is c-Met expressing cancer.

In some embodiments, the EGFR or c-Met expressing cancer is associated with a wild-type EGFR, an EGFR mutation, an EGFR gene amplification, increased levels of circulating HGF, a wild-type c-Met, a c-Met mutation, a c-Met gene amplification or a mutant KRAS. The EGFR mutation may be an activating mutation such as exon 19 deletion or L858R mutation. In some embodiments, the EGFR mutation is an EGFR exon 19 mutation. In some embodiments, the EGFR mutation is an L858R mutation.

Exemplary EGFR mutations, such as EGFR activating mutations that may be associated with cancer include point mutations, deletion mutations, insertion mutations, inversions or gene amplifications that lead to an increase in at least one biological activity of EGFR, such as elevated tyrosine kinase activity, formation of receptor homodimers and heterodimers, enhanced ligand binding etc. Mutations can be located in any portion of an EGFR gene or regulatory region associated with an EGFR gene and include mutations in exon 18, 19, 20 or 21. Other examples of EGFR activating mutations are known in the art (see e.g., U.S. Pat. Publ. No. US2005/0272083). Information about EGFR and other ErbB receptors including receptor homo- and hetero-dimers, receptor ligands, autophosphorylation sites, and signaling molecules involved in ErbB mediated signaling is known in the art (see e.g., Hynes and Lane, Nature Reviews Cancer 5: 341-354, 2005).

In some embodiments, the EGFR mutation is E709K, L718Q, L718V, G719A, G719X, G724X, G724S, I744T, E746K, L747S, E749Q, A750P, A755V, V765M, C775Y, T790M, L792H, L792V, G796S, G796R, G796C, C797S, T854I, L858P, L858R, L861X, delE746-A750, delE746_T751InsKV, delE746_A750InsHS, delE746_T751InsFPT, delE746_T751InsL, delE746_S752InsIP, delE746_P753InsMS, delE746_T751InsA, delE746_T751InsAPT, delE746_T751InsVA, delE746_S752InsV, delE746_P753InsVS, delE746_K754InsGG, delE746_E749, delE746_E749InsP, delL747_E749, delL747_A750InsP, delL747_T751InsP, delL747_T751InsN, delL747_S752InsPT, delL747_P753InsNS, delL747_S752InsPI, delL747_S752, delL747_P753InsS, delL747_K754, delL747_T751InsS, delL747_T751, delL747_P753InsS, delA750_I759InsPT, delT751_I759InsT, delS752_I759, delT751_I759InsN, delT751_D761InsNLY, delS752_I759, delR748-P753, delL747-P753insS, delL747-T751, M766_A767InsA, S768_V769InsSVA, P772_H773InsNS, D761_E762InsX, A763_Y764InsX, Y764_Y765 InsX, M766_A767InsX, A767_V768 InsX, S768_V769 InsX, V769_D770 InsX, D770_N771 InsX, N771_P772 InsX, P772_H773 InsX, H773_V774 InsX, V774_C775 InsX, one or more deletions in EGFR exon 20, or one or more insertions in EGFR exon 20, one or more deletions in EGFR exon 19, or one or more insertions in EGFR exon 19, or any combinations thereof, wherein X refers to any of the naturally occurring amino acids and can be one to seven amino acids long. In some embodiments, the EGFR mutation is L858R. The nomenclature of the mutations is well-known.

In some embodiments, the EGFR mutation is one or more deletions in exon 19 or L858R or any combination thereof. Exemplary exon 19 deletions are delE746-A750, delE746_T751InsKV, delE746_A750InsHS, delE746_T751InsFPT, delE746_T751InsL, delE746_S752InsIP, delE746_P753InsMS, delE746_T751InsA, delE746_T751InsAPT, delE746_T751InsVA, delE746_S752InsV, delE746_P753InsVS, delE746_K754InsGG, delE746_E749, delE746_E749InsP, delL747_E749, delL747_A750InsP, delL747_T751InsP, delL747_T751InsN, delL747_S752InsPT, delL747_P753InsNS, delL747_S752InsPI, delL747_S752, delL747_P753InsS, delL747_K754, delL747_T751InsS, delL747_T751, delL747_P753InsS, delA750_I759InsPT, delT751_I759InsT, delS752_I759, delT751_I759InsN, delT751_D761InsNLY, delS752_I759, delR748-P753 and delL747-P753insS, delL747-T751.

Exemplary c-Met mutations include point mutations, deletion mutations, insertion mutations, inversions or gene amplifications that lead to an increase in at least one biological activity of a c-Met protein, such as elevated tyrosine kinase activity, formation of receptor homodimers and heterodimers, enhanced ligand binding etc. Mutations can be located in any portion of the c-Met gene or regulatory regions associated with the gene, such as mutations in the kinase domain of c-Met. Exemplary c-Met mutations are mutations at residue positions N375, V13, V923, R175, V136, L229, S323, R988, S1058/T1010 and E168, or exon 14 skipping mutations. In some embodiments, the c-Met mutation is c-Met exon 14 skipping mutation.

Methods for detecting EGFR and c-Met mutations or gene amplifications are well known.

In some embodiments, the subject has been diagnosed with the EGFR mutation prior to administering the therapy comprising an EGFR/c-Met bispecific antibody.

In some embodiments, the subject has a newly diagnosed cancer. In some embodiments, the subject has a newly diagnosed EGFR or c-Met expressing cancer. In some embodiments, the subject has a newly diagnosed EGFR and c-Met expressing cancer. In some embodiments, the subject has a newly diagnosed EGFR expressing cancer. In some embodiments, the subject has a newly diagnosed c-Met expressing cancer.

In some embodiments, the subject having the newly diagnosed cancer has one or more EGFR exon 20 mutations. In some embodiments, the subject having the newly diagnosed EGFR or c-Met expressing cancer has one or more EGFR exon 20 mutations. Exon 20 mutations (insertion of one or more amino acids are generally resistant to EGFR tyrosine kinase inhibitors (TKI) (see. e.g. Int. Pat. Publ. No. WO2018/094225). Exemplary exon 20 mutations include M766_A767InsA, S768_V769InsSVA, P772_H773InsNS, D761_E762InsX, A763_Y764InsX, Y764_Y765 InsX, M766_A767InsX, A767_V768 InsX, S768_V769 InsX, V769_D770 InsX, D770_N771 InsX, N771_P772 InsX, P772_H773 InsX, H773_V774 InsX, and V774_C775 InsX, wherein X is one to seven amino acids.

In some embodiments, the subject is EGFR tyrosine kinase inhibitor (TKI) treatment naïve.

In some embodiments, the subject is resistant or relapsed to treatment with a first generation EGFR TKI.

In some embodiments, the first generation EGFR TKI is erlotinib or gefitinib.

In some embodiments, the subject is resistant or relapsed to treatment with a second generation EGFR TKI.

In some embodiments, the second generation EGFR TKI is afatinib.

In some embodiments, the subject is resistant or relapsed to treatment with a third generation EGFR TKI.

In some embodiments, the third generation EGFR TKI is osimertinib.

In some embodiments, the subject is resistant or has acquired resistance to treatment with a prior anti-cancer therapy.

In some embodiments, the prior anti-cancer therapy is chemotherapy, a targeted anti-cancer therapy or a kinase inhibitor. In some embodiments, the prior anti-cancer therapy is doublet platinum chemotherapy.

In some embodiments, the TKI is an inhibitor of EGFR, c-Met, HER2, HER3, HER4, VEGFR or AXL.

In some embodiments, the TKI is mobocertinib, erlotinib, gefitinib, lapatinib, vandetanib, afatinib, osimertinib, poziotinib, criotinib, cabozantinib, capmatinib, axitinib, lenvatinib, nintedanib, regorafenib, pazopanib, sorafenib or sunitinib.

In some embodiments, the subject is resistant or has acquired resistance to an EGFR inhibitor. Exemplary EGFR inhibitors for which cancer may acquire resistance are anti-EGFR antibodies cetuximab (ERBITUX®), pantinumumab (VECTIBIX®), matuzumab, nimotuzumab, small molecule EGFR inhibitors erlotinib (TARCEVA®), gefitinib (IRESSA®), EKB-569 (pelitinib, irreversible EGFR TKI), pan-ErbB and other receptor tyrosine kinase inhibitors, lapatinib (EGFR and HER2 inhibitor), pelitinib (EGFR and HER2 inhibitor), vandetanib (ZD6474, ZACTIMA™, EGFR, VEGFR2 and RET TKI), PF00299804 (dacomitinib, irreversible pan-ErbB TKI), CI-1033 (irreversible pan-erbB TKI), afatinib (BIBW2992, irreversible pan-ErbB TKI), AV-412 (dual EGFR and ErbB2 inhibitor), EXEL-7647 (EGFR, ErbB2, GEVGR and EphB4 inhibitor), CO-1686 (irreversible mutant-selective EGFR TKI), AZD9291 (irreversible mutant-selective EGFR TKI), and HM-272 (neratinib, irreversible EGFR/ErbB2 inhibitor).

### Anti-EGFR/c-Met Antibodies

In some embodiments, the anti-EGFR/c-Met antibody is a bispecific antibody. In certain embodiments, the antibody is an isolated antibody. In particular embodiments, the antibody is an isolated bispecific antibody.

In some embodiments, the antibody (e.g., bispecific antibody) comprises a first domain that specifically binds EGFR and a second domain that specifically binds c-Met.

### EGFR Binding Arm

In some embodiments, the first domain that specifically binds EGFR comprises:
a) heavy chain complementarity determining region 1 (HCDR1), HCDR2, HCDR3 amino acid sequences of SEQ ID NOs:1, 2 and 3, respectively; and/or
b) light chain complementarity determining region 1 (LCDR1), LCDR2 and LCDR3 amino acid sequences of SEQ ID NOs:4, 5 and 6, respectively.

In certain embodiments, the first domain that specifically binds EGFR comprises:
a) HCDR1, HCDR2, HCDR3 amino acid sequences of SEQ ID NOs:1, 2 and 3, respectively; and
b) LCDR1, LCDR2 and LCDR3 amino acid sequences of SEQ ID NOs:4, 5 and 6, respectively.

HCDR1:
(SEQ ID NO: 1)
TYGMH
HCDR2:
(SEQ ID NO: 2)
VIWDDGSYKYYGDSVKG
HCDR3:
(SEQ ID NO: 3)
DGITMVRGVMKDYFDY
LCDR1:
(SEQ ID NO: 4)
RASQDISSALV
LCDR2:
(SEQ ID NO: 5)
DASSLES
LCDR3:
(SEQ ID NO: 6)
QQFNSYPLT

In some embodiments, the first domain comprises a heavy chain variable region (VH) amino acid sequence that is at least 90% identical to SEQ ID NO:13, e.g., about: 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% identical to SEQ ID NO:13. In some embodiments, the sequence identity is about: 90-99.9%, 90-99.8%, 92-99.8%, 92-99.6%, 94-99.6%, 94-99.5%, 95-99.5%, 95-99.4%, 96-99.4%, 96-99.2%, 97-99.2% or 97-99%. In particular embodiments, the first domain comprises a VH of SEQ ID NO:13.

In certain embodiments, the first domain comprises a light chain variable region (VL) amino acid sequence that is at least 90% identical to SEQ ID NO:14, e.g., about: 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% identical to SEQ ID NO:14. In some embodiments, the sequence identity is about: 90-99.9%, 90-99.8%, 92-99.8%, 92-99.6%, 94-99.6%, 94-99.5%, 95-99.5%, 95-99.4%, 96-99.4%, 96-99.2%, 97-99.2% or 97-99%. In particular embodiments, the first domain comprises a VL of SEQ ID NO:14.

As used herein, the term “identical” or “has sequence identity,” refers to the extent to which two amino acid sequences have the same residues at the same positions when the sequences are aligned to achieve a maximal level of identity, expressed as a percentage. For sequence alignment and comparison, typically one sequence is designated as a reference sequence, to which a test sequences are compared. The sequence identity between reference and test sequences is expressed as the percentage of positions across the entire length of the reference sequence where the reference and test sequences share the same amino acid upon alignment of the reference and test sequences to achieve a maximal level of identity. As an example, two sequences are considered to have 70% sequence identity when, upon alignment to achieve a maximal level of identity, the test sequence has the same amino acid residue at 70% of the same positions over the entire length of the reference sequence.

In some embodiments, the first domain comprises:
a) a VH amino acid sequence that is at least 90% identical to SEQ ID NO:13; and/or
b) a VL amino acid sequence that is at least 90% identical to SEQ ID NO:14.

In some embodiments, the first domain comprises:
a) a VH amino acid sequence that is at least 90% identical to SEQ ID NO:13; and
b) a VL amino acid sequence that is at least 90% identical to SEQ ID NO:14.

In some embodiments, the first domain comprises:
a) a VH of SEQ ID NO:13; and/or
b) a VL of SEQ ID NO:14.

In particular embodiments, the first domain comprises:
a) a VH of SEQ ID NO:13; and
b) a VL of SEQ ID NO:14.

VH:
(SEQ ID NO: 13)
QVQLVESGGGVVQPGRSLRLSCAASGFTFSTYGMHWVRQAPGKGLE
WVAVIWDDGSYKYYGDSVKGRFTISRDNSKNTLYLQMNSLRAEDTAVYY
CARDGITMVRGVMKDYFDYWGQGTLVTVSS
VL:
(SEQ ID NO: 14)
AIQLTQSPSSLSASVGDRVTITCRASQDISSALVWYQQKPGKAPKLLIY
DASSLESGVPSRFSGSESGTDFTLTISSLQPEDFATYYCQQFNSYPLTF
GGGTKVEIK

In some embodiments, the first domain comprises a first heavy chain (HC1) amino acid sequence that is at least 80% identical to SEQ ID NO:17, e.g., about: 81%, 82%, 83%, 84%, 85%, 86%, 87%, 88%, 89%, 90%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% identical to SEQ ID NO:17. In certain embodiments, the sequence identity is about: 80-99.9%, 80-99.8%, 85-99.8%, 85-99.6%, 90-99.6%, 90-99.5%, 95-99.5%, 95-99.4%, 96-99.4%, 96-99.2%, 97-99.2% or 97-99%. In particular embodiments, the first domain comprises a HC1 amino acid sequence of SEQ ID NO:17.

In some embodiments, the first domain comprises a first light chain (LC1) amino acid sequence that is at least 80% identical to SEQ ID NO:18, e.g., about: 81%, 82%, 83%, 84%, 85%, 86%, 87%, 88%, 89%, 90%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% identical to SEQ ID NO:18. In certain embodiments, the sequence identity is about: 80-99.9%, 80-99.8%, 85-99.8%, 85-99.6%, 90-99.6%, 90-99.5%, 95-99.5%, 95-99.4%, 96-99.4%, 96-99.2%, 97-99.2% or 97-99%. In particular embodiments, the first domain comprises a LC1 amino acid sequence of SEQ ID NO:18.

In some embodiments, the first domain comprises:
a) a HC1 amino acid sequence that is at least 80% identical to SEQ ID NO:17; and/or
b) a LC1 amino acid sequence that is at least 80% identical to SEQ ID NO:18.

In some embodiments, the first domain comprises:
a) a HC1 amino acid sequence that is at least 80% identical to SEQ ID NO:17; and
b) a LC1 amino acid sequence that is at least 80% identical to SEQ ID NO:18.

In some embodiments, the first domain comprises:
a) a HC1 of SEQ ID NO:17; and/or
b) a LC1 of SEQ ID NO:18.

In some embodiments, the first domain comprises:
a) a HC1 of SEQ ID NO:17; and
b) a LC1 of SEQ ID NO:18.

HC1:
(SEQ ID NO: 17)
QVQLVESGGGVVQPGRSLRLSCAASGFTFSTYGMHWVRQAPGKGL
EWVAVIWDDGSYKYYGDSVKGRFTISRDNSKNTLYLQMNSLRAEDTAVY
YCARDGITMVRGVMKDYFDYWGQGTLVTVSSASTKGPSVFPLAPSSKST
SGGTAALGCLVKDYFPEPVTVSWNSGALTSGVHTFPAVLQSSGLYSLSS
VVTVPSSSLGTQTYICNVNHKPSNTKVDKRVEPKSCDKTHTCPPCPAPE
LLGGPSVFLFPPKPKDTLMISRTPEVTCVVVDVSHEDPEVKFNWYVDGV
EVHNAKTKPREEQYNSTYRVVSVLTVLHQDWLNGKEYKCKVSNKALPAP
IEKTISKAKGQPREPQVYTLPPSREEMTKNQVSLTCLVKGFYPSDIAVE
WESNGQPENNYKTTPPVLDSDGSFLLYSKLTVDKSRWQQGNVFSCSVMH
EALHNHYTQKSLSLSPGK
LC1:
(SEQ ID NO: 18)
AIQLTQSPSSLSASVGDRVTITCRASQDISSALVWYQQKPGKAPKLLI
YDASSLESGVPSRFSGSESGTDFTLTISSLQPEDFATYYCQQFNSYPLT
FGGGTKVEIKRTVAAPSVFIFPPSDEQLKSGTASVVCLLNNFYPREAKV
QWKVDNALQSGNSQESVTEQDSKDSTYSLSSTLTLSKADYEKHKVYACE
VTHQGLSSPVTKSFNRGEC

c-Met Binding Arm

In certain embodiments, the second domain that specifically binds c-Met comprises:
a) HCDR1, HCDR2, HCDR3 amino acid sequences of SEQ ID NOs:7, 8 and 9, respectively; and/or
b) LCDR1, LCDR2 and LCDR3 amino acid sequences of SEQ ID NOs:10, 11 and 12, respectively.

In certain embodiments, the second domain that specifically binds c-Met comprises:
a) HCDR1, HCDR2, HCDR3 amino acid sequences of SEQ ID NOs:7, 8 and 9, respectively; and
b) LCDR1, LCDR2 and LCDR3 amino acid sequences of SEQ ID NOs:10, 11 and 12, respectively.

HCDR1:
(SEQ ID NO: 7)
SYGIS
HCDR2:
(SEQ ID NO: 8)
WISAYNGYTNYAQKLQG
HCDR3:
(SEQ ID NO: 9)
DLRGTNYFDY
LCDR1:
(SEQ ID NO: 10)
RASQGISNWLA
LCDR2:
(SEQ ID NO: 11)
AASSLLS
LCDR3:
(SEQ ID NO: 12)
QQANSFPIT

In some embodiments, the second domain comprises a VH amino acid sequence that is at least 90% identical to SEQ ID NO:15, e.g., about: 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% identical to SEQ ID NO:15. In some embodiments, the sequence identity is about: 90-99.9%, 90-99.8%, 92-99.8%, 92-99.6%, 94-99.6%, 94-99.5%, 95-99.5%, 95-99.4%, 96-99.4%, 96-99.2%, 97-99.2% or 97-99%. In particular embodiments, the second domain comprises a VH of SEQ ID NO:15

In certain embodiments, the second domain comprises a VL amino acid sequence that is at least 90% identical to SEQ ID NO:16, e.g., about: 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% identical to SEQ ID NO:16. In some embodiments, the sequence identity is about: 90-99.9%, 90-99.8%, 92-99.8%, 92-99.6%, 94-99.6%, 94-99.5%, 95-99.5%, 95-99.4%, 96-99.4%, 96-99.2%, 97-99.2% or 97-99%. In particular embodiments, the second domain comprises a VL of SEQ ID NO:16.

In some embodiments, the second domain comprises:
a) a VH amino acid sequence that is at least 90% identical to SEQ ID NO:15; and/or
b) a VL amino acid sequence that is at least 90% identical to SEQ ID NO:16.

In some embodiments, the second domain comprises:
a) a VH amino acid sequence that is at least 90% identical to SEQ ID NO:15; and
b) a VL amino acid sequence that is at least 90% identical to SEQ ID NO:16.

In some embodiments, the second domain comprises:
a) a VH of SEQ ID NO:15; and/or
b) a VL of SEQ ID NO:16.

In particular embodiments, the second domain comprises:
a) a VH of SEQ ID NO:15; and
b) a VL of SEQ ID NO:16.

VH:
(SEQ ID NO: 15)
QVQLVQSGAEVKKPGASVKVSCETSGYTFTSYGISWVRQAPGHGLE
WMGWISAYNGYTNYAQKLQGRVTMTTDTSTSTAYMELRSLRSDDTAVYY
CARDLRGTNYFDYWGQGTLVTVSS
VL:
(SEQ ID NO: 16)
DIQMTQSPSSVSASVGDRVTITCRASQGISNWLAWFQHKPGKAPKLLI
YAASSLLSGVPSRFSGSGSGTDFTLTISSLQPEDFATYYCQQANSFPIT
FGQGTRLEIK

In some embodiments, the second domain comprises a second heavy chain (HC2) amino acid sequence that is at least 80% identical to SEQ ID NO:19, e.g., about: 81%, 82%, 83%, 84%, 85%, 86%, 87%, 88%, 89%, 90%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% identical to SEQ ID NO:19. In certain embodiments, the sequence identity is about: 80-99.9%, 80-99.8%, 85-99.8%, 85-99.6%, 90-99.6%, 90-99.5%, 95-99.5%, 95-99.4%, 96-99.4%, 96-99.2%, 97-99.2% or 97-99%. In particular embodiments, the second domain comprises a HC2 amino acid sequence of SEQ ID NO:19.

In some embodiments, the second domain comprises a second light chain (LC2) amino acid sequence that is at least 80% identical to SEQ ID NO:20, e.g., about: 81%, 82%, 83%, 84%, 85%, 86%, 87%, 88%, 89%, 90%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% identical to SEQ ID NO:20. In certain embodiments, the sequence identity is about: 80-99.9%, 80-99.8%, 85-99.8%, 85-99.6%, 90-99.6%, 90-99.5%, 95-99.5%, 95-99.4%, 96-99.4%, 96-99.2%, 97-99.2% or 97-99%. In particular embodiments, the second domain comprises a LC2 amino acid sequence of SEQ ID NO:20.

In some embodiments, the second domain comprises:
a) a HC2 amino acid sequence that is at least 80% identical to SEQ ID NO:19; and/or
b) a LC2 amino acid sequence that is at least 80% identical to SEQ ID NO:20.

In some embodiments, the second domain comprises:
a) a HC2 amino acid sequence that is at least 80% identical to SEQ ID NO:19; and
b) a LC2 amino acid sequence that is at least 80% identical to SEQ ID NO:20.

In some embodiments, the second domain comprises:
a) a HC2 of SEQ ID NO:19; and/or
b) a LC2 of SEQ ID NO:20.

In some embodiments, the second domain comprises:
a) a HC2 of SEQ ID NO:19; and
b) a LC2 of SEQ ID NO:20.

HC2:
(SEQ ID NO: 19)
QVQLVQSGAEVKKPGASVKVSCETSGYTFTSYGISWVRQAPGHGLE
WMGWISAYNGYTNYAQKLQGRVTMTTDTSTSTAYMELRSLRSDDTAVYY
CARDLRGTNYFDYWGQGTLVTVSSASTKGPSVFPLAPSSKSTSGGTAAL
GCLVKDYFPEPVTVSWNSGALTSGVHTFPAVLQSSGLYSLSSVVTVPSS
SLGTQTYICNVNHKPSNTKVDKRVEPKSCDKTHTCPPCPAPELLGGPSV
FLFPPKPKDTLMISRTPEVTCVVVDVSHEDPEVKFNWYVDGVEVHNAKT
KPREEQYNSTYRVVSVLTVLHQDWLNGKEYKCKVSNKALPAPIEKTISK
AKGQPREPQVYTLPPSREEMTKNQVSLTCLVKGFYPSDIAVEWESNGQP
ENNYKTTPPVLDSDGSFFLYSRLTVDKSRWQQGNVFSCSVMHEALHNHY
TQKSLSLSPGK
LC2:
(SEQ ID NO: 20)
DIQMTQSPSSVSASVGDRVTITCRASQGISNWLAWFQHKPGKAPKLL
IYAASSLLSGVPSRFSGSGSGTDFTLTISSLQPEDFATYYCQQANSFPI
TFGQGTRLEIKRTVAAPSVFIFPPSDEQLKSGTASVVCLLNNFYPREAK
VQWKVDNALQSGNSQESVTEQDSKDSTYSLSSTLTLSKADYEKHKVYAC
EVTHQGLSSPVTKSFNRGEC

In some embodiments, the antibody (e.g., bispecific antibody) comprises:
a) a first domain that specifically binds EGFR, comprising HCDR1, HCDR2, HCDR3, LCDR1, LCDR2 and LCDR3 amino acid sequences of SEQ ID NOs:1, 2, 3, 4, 5 and 6, respectively; and/or
b) a second domain that specifically binds c-Met, comprising HCDR1, HCDR2, HCDR3, LCDR1, LCDR2 and LCDR3 amino acid sequences of SEQ ID NOs:7, 8, 9, 10, 11 and 12, respectively.

In certain embodiments, the antibody (e.g., bispecific antibody) comprises:
a) a first domain that specifically binds EGFR, comprising HCDR1, HCDR2, HCDR3, LCDR1, LCDR2 and LCDR3 amino acid sequences of SEQ ID NOs:1, 2, 3, 4, 5 and 6, respectively; and
b) a second domain that specifically binds c-Met, comprising HCDR1, HCDR2, HCDR3, LCDR1, LCDR2 and LCDR3 amino acid sequences of SEQ ID NOs:7, 8, 9, 10, 11 and 12, respectively.

In some embodiments, the antibody (e.g., bispecific antibody) comprises:
a) a first domain comprising a VH amino acid sequence that is at least 90% identical to SEQ ID NO:13;
b) a first domain comprising a VL amino acid sequence that is at least 90% identical to SEQ ID NO:14;
c) a second domain comprising a VH amino acid sequence that is at least 90% identical to SEQ ID NO:15; and/or
d) a second domain comprising a VL amino acid sequence that is at least 90% identical to SEQ ID NO:16.

In certain embodiments, the antibody (e.g., bispecific antibody) comprises:
a) a first domain comprising a VH amino acid sequence that is at least 90% identical to SEQ ID NO:13;
b) a first domain comprising a VL amino acid sequence that is at least 90% identical to SEQ ID NO:14;
c) a second domain comprising a VH amino acid sequence that is at least 90% identical to SEQ ID NO:15; and
d) a second domain comprising a VL amino acid sequence that is at least 90% identical to SEQ ID NO:16.

In some embodiments, the antibody (e.g., bispecific antibody) comprises:
a) a first domain comprising a VH of SEQ ID NO:13;
b) a first domain comprising a VL of SEQ ID NO:14;
c) a second domain comprising a VH of SEQ ID NO:15; and/or
d) a second domain comprising a VL of SEQ ID NO:16.

In some embodiments, the antibody (e.g., bispecific antibody) comprises:
a) a first domain comprising a VH of SEQ ID NO:13;
b) a first domain comprising a VL of SEQ ID NO:14;
c) a second domain comprising a VH of SEQ ID NO:15; and
d) a second domain comprising a VL of SEQ ID NO:16.

In certain embodiments, the antibody (e.g., bispecific antibody) comprises:
a) a HC1 amino acid sequence that is at least 80% identical to SEQ ID NO:17;
b) a LC1 amino acid sequence that is at least 80% identical to SEQ ID NO:18;
c) a HC2 amino acid sequence that is at least 80% identical to SEQ ID NO:19; and/or
d) a LC2 amino acid sequence that is at least 80% identical to SEQ ID NO:20.

In certain embodiments, the antibody (e.g., bispecific antibody) comprises:
a) a HC1 amino acid sequence that is at least 80% identical to SEQ ID NO:17;
b) a LC1 amino acid sequence that is at least 80% identical to SEQ ID NO:18;
c) a HC2 amino acid sequence that is at least 80% identical to SEQ ID NO:19; and
d) a LC2 amino acid sequence that is at least 80% identical to SEQ ID NO:20.

In certain embodiments, the antibody (e.g., bispecific antibody) comprises:
a) a HC1 of SEQ ID NO:17;
b) a LC1 of SEQ ID NO:18;
c) a HC2 of SEQ ID NO:19; and/or
d) a LC2 of SEQ ID NO:20.

In particular embodiments, the antibody (e.g., bispecific antibody) comprises:
a) a HC1 of SEQ ID NO:17;
b) a LC1 of SEQ ID NO:18;
c) a HC2 of SEQ ID NO:19; and
d) a LC2 of SEQ ID NO:20.

In some embodiments, the antibody (e.g., bispecific antibody) is of the IgG isotype. In certain embodiments, the antibody (e.g., bispecific antibody) is of the IgG1 isotype. Some variation exists within the IgG1 constant domain (e.g., well-known allotypes), for example, with variation at positions 214, 356, 358, 422, 431, 435 and/or 436 (residue numbering according to the EU numbering) (see e.g., IMGT Web resources; IMGT Repertoire (IG and TR); Proteins and alleles; allotypes). The bispecific anti-EGFR/c-Met antibody may be of any IgG1 allotype, such as G1m17, G1m3, G1m1, G1m2, G1m27 or G1m28.

In some embodiments, the antibody is a human antibody.

In particular embodiments, the antibody is amivantamab. Amivantamab or JNJ-61186372 (JNJ-372) is an IgG1 anti-EGFR/c-Met bispecific antibody described in U.S. Pat. No. 9,593,164.

Other anti-EGFR/c-Met antibodies (e.g., bispecific antibodies) may also be used in the methods of the disclosure, for example, by combining publicly available EGFR binding VH/VL domains and c-Met binding VH/VL domains.

In some embodiments, the antibody (e.g., bispecific antibody) comprises a biantennary glycan structure with a fucose content of between about 1% to about 15%.

Antibodies with reduced fucose content can be made using different methods reported to lead to the successful expression of relatively high defucosylated antibodies bearing the biantennary complex-type of Fc oligosaccharides such as control of culture osmolality (Konno et al., Cytotechnology 64(:249-65, 2012), application of a variant CHO line Lec13 as the host cell line (Shields et al., J Biol Chem 277:26733-26740, 2002), application of a variant CHO line EB66 as the host cell line (Olivier et al., MAbs; 2(4), 2010; Epub ahead of print; PMID:20562582), application of a rat hybridoma cell line YB2/0 as the host cell line (Shinkawa et al., J Biol Chem 278:3466-3473, 2003), introduction of small interfering RNA specifically against the α 1,6-fucosyltrasferase (FUT8) gene (Mori et al., Biotechnol Bioeng 88:901-908, 2004), or coexpression of β-1,4-N-acetylglucosaminyltransferase III and Golgi α-mannosidase II or a potent alpha-mannosidase I inhibitor, kifunensine (Ferrara et al., J Biol Chem 281:5032-5036, 2006, Ferrara et al., Biotechnol Bioeng 93:851-861, 2006; Zhou et al., Biotechnol Bioeng 99:652-65, 2008). In general, lowering fucose content in the glycan of the antibodies potentiates antibody-mediated cellular cytotoxicity (ADCC).

### Generating Anti-EGFR/c-Met Antibodies

Anti-EGFR/c-Met antibodies used in the methods of the disclosure may be generated, for example, using Fab arm exchange (or half molecule exchange) between two monospecific bivalent antibodies by introducing substitutions at the heavy chain CH3 interface in each half molecule to favor heterodimer formation of two antibody half molecules having distinct specificity either in vitro in cell-free environment or using co-expression. The Fab arm exchange reaction is the result of a disulfide-bond isomerization reaction and dissociation-association of CH3 domains. The heavy chain disulfide bonds in the hinge regions of the parental monospecific antibodies are reduced. The resulting free cysteines of one of the parental monospecific antibodies form an inter heavy-chain disulfide bond with cysteine residues of a second parental monospecific antibody molecule and simultaneously CH3 domains of the parental antibodies release and reform by dissociation-association. The CH3 domains of the Fab arms may be engineered to favor heterodimerization over homodimerization. The resulting product is a bispecific antibody having two Fab arms or half molecules which each bind a distinct epitope, i.e., an epitope on EGFR and an epitope on c-Met. For example, the bispecific antibodies of the invention may be generated using the technology described in Int. Pat. Publ. No. WO2011/131746. Mutations F405L in one heavy chain and K409R in the other heavy chain may be used in case of IgG1 antibodies. For IgG2 antibodies, a wild-type IgG2 and a IgG2 antibody with F405L and R409K substitutions may be used. For IgG4 antibodies, a wild-type IgG4 and a IgG4 antibody with F405L and R409K substitutions may be used. To generate bispecific antibodies, first monospecific bivalent antibody and the second monospecific bivalent antibody are engineered to have the aforementioned mutation in the Fc region, the antibodies are incubated together under reducing conditions sufficient to allow the cysteines in the hinge region to undergo disulfide bond isomerization; thereby generating the bispecific antibody by Fab arm exchange. The incubation conditions may optimally be restored to non-reducing. Exemplary reducing agents that may be used are 2-mercaptoethylamine (2-MEA), dithiothreitol (DTT), dithioerythritol (DTE), glutathione, tris(2-carboxyethyl)phosphine (TCEP), L-cysteine and beta-mercaptoethanol. For example, incubation for at least 90 min at a temperature of at least 20° C. in the presence of at least 25 mM 2-MEA or in the presence of at least 0.5 mM dithiothreitol at a pH of from 5-8, for example at pH of 7.0 or at pH of 7.4 may be used.

Bispecific anti-EGFR/c-Met antibodies used in the methods of the disclosure may also be generated using designs such as the Knob-in-Hole (Genentech), CrossMAbs (Roche) and the electrostatically-matched (Chugai, Amgen, NovoNordisk, Oncomed), the LUZ-Y (Genentech), the Strand Exchange Engineered Domain body (SEEDbody) (EMD Serono), and the Biclonic (Merus).

In the “knob-in-hole” strategy (see, e.g., Intl. Publ. No. WO 2006/028936) select amino acids forming the interface of the CH3 domains in human IgG can be mutated at positions affecting CH3 domain interactions to promote heterodimer formation. An amino acid with a small side chain (hole) is introduced into a heavy chain of an antibody specifically binding a first antigen and an amino acid with a large side chain (knob) is introduced into a heavy chain of an antibody specifically binding a second antigen. After co-expression of the two antibodies, a heterodimer is formed as a result of the preferential interaction of the heavy chain with a “hole” with the heavy chain with a “knob”. Exemplary CH3 substitution pairs forming a knob and a hole are (expressed as modified position in the first CH3 domain of the first heavy chain/modified position in the second CH3 domain of the second heavy chain): T366Y/F405A, T366W/F405W, F405W/Y407A, T394W/Y407T, T394S/Y407A, T366W/T394S, F405W/T394S and T366W/T366S L368A_Y407V.

CrossMAb technology, in addition to utilizing the “knob-in-hole” strategy to promoter Fab arm exchange utilizes CH1/CL domain swaps in one half arm to ensure correct light chain pairing of the resulting bispecific antibody (see e.g., U.S. Pat. No. 8,242,247).

Other cross-over strategies may be used to generate full length bispecific antibodies of the invention by exchanging variable or constant, or both domains between the heavy chain and the light chain or within the heavy chain in the bispecific antibodies, either in one or both arms. These exchanges include for example VH-CH1 with VL-CL, VH with VL, CH3 with CL and CH3 with CH1 as described in Int. Patent Publ. Nos. WO2009/080254, WO2009/080251, WO2009/018386 and WO2009/080252.

Other strategies such as promoting heavy chain heterodimerization using electrostatic interactions by substituting positively charged residues at one CH3 surface and negatively charged residues at a second CH3 surface may be used, as described in US Patent Publ. No. US2010/0015133; US Patent Publ. No. US2009/0182127; US Patent Publ. No. US2010/028637 or US Patent Publ. No. US2011/0123532. In other strategies, heterodimerization may be promoted by following substitutions (expressed as modified positions in the first CH3 domain of the first heavy chain/modified position in the second CH3 domain of the second heavy chain): L351Y_F405A_Y407V/T394W, T366I_K392M_T394W/F405A_Y407V, T366L_K392M_T394W/F405A_Y407V, L351Y_Y407A/T366A_K409F, L351Y_Y407A/T366V_K409F, Y407A/T366A_K409F, or T350V_L351Y_F405A_Y407V/T350V_T366L_K392L_T394W as described in U.S. Patent Publ. No. US2012/0149876 or U.S. Patent Publ. No. US2013/0195849.

SEEDbody technology may be utilized to generate bispecific antibodies of the invention. SEEDbodies have, in their constant domains, select IgG residues substituted with IgA residues to promote heterodimerization as described in U.S. Patent No. US20070287170.

Mutations are typically made at the DNA level to a molecule such as the constant domain of the antibody using standard methods.

### Administration

The anti-EGFR/c-Met antibody (e.g., bispecific antibody) may be administered in a pharmaceutical composition. In some embodiments, the pharmaceutical composition further comprises a pharmaceutically acceptable carrier.

In some embodiments, the pharmaceutical composition comprising the anti-EGFR/c-Met antibody (e.g., bispecific antibody) is administered via an intravenous infusion.

In some embodiments, the pharmaceutical composition comprising the anti-EGFR/c-Met antibody is administered subcutaneously (SC).

In some embodiments, the antibody is administered at a dose of about 700 mg to about 2,240 mg. In some embodiments, the antibody is administered at a dose of about 700 mg, about 1,050 mg about 1,400 mg, about 1,600 mg, or about 2,240 mg. In some embodiments, the antibody is administered at a dose of about 1,050 mg. In certain embodiments, the antibody is administered at a dose of about 1,400 mg. In particular embodiments, the antibody is administered at a dose of about 700 mg. In some embodiments, the antibody is administered at a dose of about 1,600 mg. In some embodiments, the antibody is administered at a dose of about 2,240 mg.

In some embodiments, the antibody is administered at a dose of about 350 mg.

In some embodiments, the antibody is administered at a dose of about 750 mg.

In some embodiments, the antibody is administered at a dose of about 800 mg.

In some embodiments, the antibody is administered at a dose of about 850 mg.

In some embodiments, the antibody is administered at a dose of about 900 mg.

In some embodiments, the antibody is administered at a dose of about 950 mg.

In some embodiments, the antibody is administered at a dose of about 1,000 mg.

In some embodiments, the antibody is administered at a dose of about 1,100 mg.

In some embodiments, the antibody is administered at a dose of about 1,150 mg.

In some embodiments, the antibody is administered at a dose of about 1,200 mg.

In some embodiments, the antibody is administered at a dose of about 1,250 mg.

In some embodiments, the antibody is administered at a dose of about 1,300 mg.

In some embodiments, the antibody is administered at a dose of about 1,350 mg.

In some embodiments, the antibody is administered at a dose of about 1,500 mg.

In some embodiments, the antibody is administered at a dose of about 1,600 mg.

In some embodiments, the antibody is administered at a dose of about 1,700 mg.

In some embodiments, the antibody is administered at a dose of about 1,800 mg.

In some embodiments, the antibody is administered at a dose of about 1,900 mg.

In some embodiments, the antibody is administered at a dose of about 2,000 mg.

In some embodiments, the antibody is administered at a dose of about 2,100 mg.

In some embodiments, the antibody is administered at a dose of about 2,200 mg.

In some embodiments, the antibody is administered at a dose of about 2,240 mg.

In some embodiments, the antibody is administered at a dose of about 2,300 mg.

In certain embodiments, the antibody is administered at a dose of 1,050 mg for body weight <80 kg and 1,400 mg for body weight ≥80 kg.

In particular embodiments, the antibody is administered at a dose of 700 mg for body weight <80 kg and 1,050 mg for body weight ≥80 kg.

In certain embodiments, the antibody is administered at a dose of 1,600 mg for body weight <80 kg and 2,240 mg for body weight ≥80 kg.

In some embodiments, the antibody is administered twice a week.

In certain embodiments, the antibody is administered once a week.

In some embodiments, the antibody is administered once every two weeks.

In certain embodiments, the antibody is administered once every three weeks.

In some embodiments, the antibody is administered once every four weeks.

In certain embodiments, the antibody is administered once a week or once every two weeks. In particular embodiments, the antibody is administered once weekly for the first 4 weeks and then every 2 weeks.

In some embodiments, the antibody is administered on a 28-day cycle.

In some embodiments, the subject has a body weight of <80 kg, and the antibody (e.g., bispecific antibody such as amivantamab) is administered at a dose of 700 mg once weekly for the first 4 weeks and then every 2 weeks 28-day cycles. In other embodiments, the subject has a body weight of <80 kg, and the antibody (e.g., bispecific antibody such as amivantamab) is administered at a dose of 1,050 mg once weekly for the first 4 weeks and then every 2 weeks 28-day cycles. In other embodiments, the subject has a body weight of <80 kg, and the antibody (e.g., bispecific antibody such as amivantamab) is administered at a dose of 1,600 mg once weekly for the first 4 weeks and then every 2 weeks 28-day cycles.

In certain embodiments, the subject has a body weight of ≥80 kg, and the antibody (e.g., bispecific antibody such as amivantamab) is administered at a dose of 1,050 mg once weekly for the first 4 weeks and then every 2 weeks 28-day cycles. In other embodiments, the subject has a body weight of ≥80 kg, and the antibody (e.g., bispecific antibody such as amivantamab) is administered at a dose of 1,400 mg once weekly for the first 4 weeks and then every 2 weeks 28-day cycles. In other embodiments, the subject has a body weight of ≥80 kg, and the antibody (e.g., bispecific antibody such as amivantamab) is administered at a dose of 2,240 mg once weekly for the first 4 weeks and then every 2 weeks 28-day cycles.

Pharmaceutical compositions comprising 1,400 mg, 1,050 mg and 700 mg dose of the anti-EGFR/c-Met antibody can be administered in total volumes of about 28 mL, 21 mL and 14 mL, respectively, with 350 mg/7 mL (50 mg/mL) solution in a single-dose vial.

Additional information regarding amivantamab can be found, for example, in the prescribing information product insert for RYBREVANT® (amivantamab-vmjw) (www.janssenlabels.com/package-insert/product-monograph/prescribing-information/RYBREVANT-pi.pdf), which is incorporated herein by reference.

In some embodiments, the antibody is administered as a monotherapy.

### Additional Therapeutic Agents

In one aspect, the disclosure provides a method of reducing occurrence or severity of infusion-related reactions (IRRs) in a subject treated with a combination treatment, comprising an anti-epidermal growth factor receptor (EGFR)/hepatocyte growth factor receptor (c-Met) antibody, comprising administering one or more of methotrexate, montelukast, or dexamethasone.

In some embodiments, the combination treatment, comprising an EGFR/c-Met bispecific antibody, also comprises one or more anti-cancer therapies comprising one or more chemotherapeutic agents, checkpoint inhibitors, targeted anti-cancer therapies or kinase inhibitors, or any combination thereof.

In some embodiments, the kinase inhibitor is an inhibitor of EGFR, an inhibitor of c-Met, an inhibitor of HER2, an inhibitor of HER3, an inhibitor of HER4, an inhibitor of VEGFR or an inhibitor of AXL.

In some embodiments, the kinase inhibitor is erlotinib, gefitinib, lapatinib, vandetanib, afatinib, osimertinib, lazertinib, mobocertinib, poziotinib, criotinib, cabozantinib, capmatinib, axitinib, lenvatinib, nintedanib, regorafenib, pazopanib, sorafenib or sunitinib. In some embodiments, the kinase inhibitor is Lazertinib. In some embodiments, the kinase inhibitor is osimertinib. In some embodiments, the kinase inhibitor is mobocertinib.

In some embodiments, the one or more prior anti-cancer therapies comprises carboplatin, paclitaxel, gemcitabine, cisplatin, vinorelbine, docetaxel, palbociclib, crizotinib, PD-(L)1 axis inhibitor, an inhibitor of EGFR, an inhibitor of c-Met, an inhibitor of HER2, an inhibitor of HER3, an inhibitor of HER4, an inhibitor of VEGFR, an inhibitor of AXL, erlotinib, gefitinib, lapatinib, vandetanib, afatinib, osimertinib, lazertinib, mobocertinib, poziotinib, criotinib, cabozantinib, capmatinib, axitinib, lenvatinib, nintedanib, regorafenib, pazopanib, sorafenib or sunitinib, or any combination thereof.

Lazertinib is a 3rd generation EGFR tyrosine kinase inhibitor (TKI); the structure and synthesis of lazertinib is described in U.S. Pat. No. 9,593,098, which is incorporated by reference herein. The chemical name of the lazertinib free base, which is represented by formula (I) herein, is N-(5-(4-(4-((dimethylamino)methyl)-3-phenyl-1H-pyrazol-1-yl)pyrimidin-2-ylamino)-4-methoxy-2-morpholinophenyl)acrylamide (referred to herein as lazertinib). The mesylate salt of lazertinib may be represented by formula II:

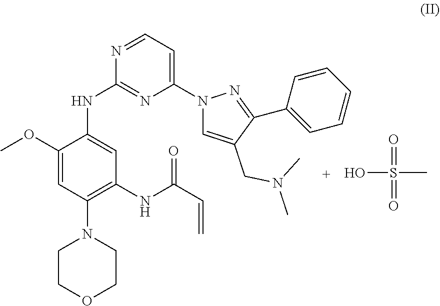

Embodiments of lazertinib (e.g., salts and crystalline forms) are described in PCT/KR2018/004473, which is also incorporated by reference herein.

According to particular embodiments, lazertinib in the form of a free base has little to no effect on wild-type EGFR, and is a highly selective and irreversible EGFR TKI with strong inhibitory activity against the single mutation of T790M and dual mutations, e.g., it targets the activating EGFR mutations del19 and L858R, as well as the T790M mutation. In one aspect of the invention, the mutation may be delE746-A750, L858R, or T790M, and it may be dual mutations selected from delE746-A750/T790M or L858R/T790M.

An embodiment of the disclosure provides a method of treating a subject having a cancer, comprising administering to the subject a combination therapy, wherein the combination therapy comprises a therapeutically effective amount of an isolated bispecific anti-epidermal growth factor receptor (EGFR)/hepatocyte growth factor receptor (c-Met) antibody and a therapeutically effective amount of a compound of formula (I):

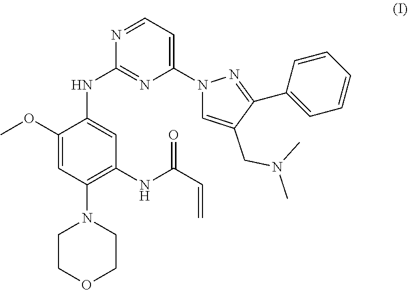 [SMILES: C=CC(=O)Nc1cc(Nc2nccc(-n3cc(CN(C)C)c(-c4ccccc4)n3)n2)c(OC)cc1N1CCOCC1]

or solvate, hydrate, tautomer, or a pharmaceutically acceptable salt thereof.

An embodiment of the disclosure provides a pharmaceutical combination comprising a therapeutically effective amount of an isolated bispecific anti-epidermal growth factor receptor (EGFR)/hepatocyte growth factor receptor (c-Met) antibody and a therapeutically effective amount of a compound of formula (I), or solvate, hydrate, tautomer, or a pharmaceutically acceptable salt thereof, for use as a medicament, in particular for use as a medicament in a subject.

In each embodiment, the bispecific anti-EGFR/c-Met antibody and the lazertinib compound, or solvate, hydrate, tautomer, or a pharmaceutically acceptable salt thereof, may be administered at the same time (e.g., as part of the same pharmaceutical composition, or in separate pharmaceutical compositions) or at different times, as described herein.

Pharmaceutically acceptable salt forms include pharmaceutically acceptable acidic/anionic or basic/cationic salts. Pharmaceutically acceptable acidic/anionic salts include acetate, benzenesulfonate, benzoate, bicarbonate, bitartrate, bromide, calcium edetate, camsylate, carbonate, chloride, citrate, dihydrochloride, edetate, edisylate, estolate, esylate, fumarate, glyceptate, gluconate, glutamate, glycollylarsanilate, hexylresorcinate, hydrobromide, hydrochloride, hydroxynaphthoate, iodide, isethionate, lactate, lactobionate, malate, maleate, malonate, mandelate, mesylate, methylsulfate, mucate, napsylate, nitrate, pamoate, pantothenate, phosphate/diphosphate, polygalacturonate, salicylate, stearate, subacetate, succinate, sulfate, hydrogensulfate, tannate, tartrate, teoclate, tosylate, and triethiodide salts. Pharmaceutically acceptable basic/cationic salts include, the sodium, potassium, calcium, magnesium, diethanolamine, N-methyl-D-glucamine, L-lysine, L-arginine, ammonium, ethanolamine, piperazine and triethanolamine salts.

A pharmaceutically acceptable acid salt is formed by reaction of the free base form of a compound of Formula (I) with a suitable inorganic or organic acid including, but not limited to, hydrobromic, hydrochloric, sulfuric, nitric, phosphoric, succinic, maleic, formic, acetic, propionic, fumaric, citric, tartaric, lactic, benzoic, salicylic, glutamic, aspartic, p-toluenesulfonic, benzenesulfonic, methanesulfonic, ethanesulfonic, naphthalenesulfonic such as 2-naphthalenesulfonic, or hexanoic acid. A pharmaceutically acceptable acid addition salt of a compound of Formula (I) can comprise or be, for example, a hydrobromide, hydrochloride, sulfate, nitrate, phosphate, succinate, maleate, formarate, acetate, propionate, fumarate, citrate, tartrate, lactate, benzoate, salicylate, glutamate, aspartate, p-toluenesulfonate, benzenesulfonate, methanesulfonate, ethanesulfonate, naphthalenesulfonate (e.g., 2-naphthalenesulfonate) or hexanoate salt.

The free acid or free base forms of the compound of formula (I) may be prepared from the corresponding base addition salt or acid addition salt form, respectively. For example, a compound of the invention in an acid addition salt form may be converted to the corresponding free base form by treating with a suitable base (e.g., ammonium hydroxide solution, sodium hydroxide, and the like). A compound of the invention in a base addition salt form may be converted to the corresponding free acid by treating with a suitable acid (e.g., hydrochloric acid, etc.).

In certain embodiments, the method further comprises administering to the subject one or more additional therapeutic agents. Non-limiting examples of the one or more additional therapeutic agents include a T cell expressing chimeric antigen receptor (CAR) (CAR-T cell), a natural killer cell expressing CAR (CAR-NK cell), a macrophage expressing CAR (CAR-M cell), a chemotherapeutic agent, an immune checkpoint inhibitor, a T-cell redirector, radiation therapy, surgery and a standard of care drug. In certain embodiments, the one or more additional therapeutic agents comprises chemotherapy, radiation therapy, surgery, a targeted anti-cancer therapy, a kinase inhibitor, or a combination thereof.

In some embodiments, the one or more additional therapeutic agents are one or more anti-cancer therapies. In some embodiments, the one or more additional therapeutic agents comprise one or more chemotherapeutic agents.

A non-exhaustive list of chemotherapeutic agents considered for use in combination therapies include anastrozole (Arimidex®), bicalutamide (Casodex®), bleomycin sulfate (Blenoxane), busulfan (Mylerae), leucovorin calcium, melphalan (Alkeran®), 6-mercaptopurine (Purinethol®), methotrexate (Folex®), mitoxantrone (Novantrone®), mylotarg, paclitaxel (Taxol®), phoenix (Yttrium90/MX-DTPA), pentostatin, polifeprosan 20 with carmustine implant (Gliadel®), dactinomycin (Actinomycin D, Cosmegan), daunorubicin hydrochloride (Cerubidine®), daunorubicin citrate liposome injection (DaunoXome®), dexamethasone, docetaxel (Taxotere®), doxorubicin hydrochloride (Adriamycin®, Rubex®), etoposide (Vepesid®), busulfan injection (Busulfex®), capecitabine (Xeloda®), N4-pentoxycarbonyl-5-deoxy-5-fluorocytidine, carboplatin (Paraplatie), carmustine (BiCNU®), chlorambucil (Leukeran®), cisplatin (Platinol®), cladribine (Leustatin®), cyclophosphamide (Cytoxan® or Neosar®), cytarabine, cytosine arabinoside (Cytosar-U®), cytarabine liposome injection (DepoCyt®), dacarbazine (DTIC-Dome®), fludarabine phosphate (Fludara®) 5-fluorouracil (Adrucil®, Efudex®), flutamide (Eulexin®), tezacitibine, Gemcitabine (difluorodeoxycitidine), hydroxyurea (Hydrea®), Idarubicin (Idamycie), ifosfamide (IFEX®), irinotecan (Camptosar®), L-asparaginase (ELSPAR®), tamoxifen citrate (Nolvadex®), teniposide (Vumon®), 6-thioguanine, thiotepa, tirapazamine (Tirazone®), topotecan hydrochloride for injection (Hycamptin®), vinblastine (Velban®), vincristine (Oncovin®), and vinorelbine (Navelbine®).

Example alkylating agents include, without limitation, nitrogen mustards, ethylenimine derivatives, alkyl sulfonates, nitrosoureas and triazenes): uracil mustard (Aminouracil Mustard®, Chlorethaminacil®, Haemanthamine®, Nordopan®, Uracil Nitrogen Mustard®, Uracillost®, Uracilmostaza®, Uramustin®, Uramustine®), chlormethine (Mustargen®), cyclophosphamide (Cytoxan®, Neosar®, Clafen®, Endoxan®, Procytox®, Revimmune™), ifosfamide (Mitoxana®), melphalan (Alkeran®), Chlorambucil (Leukeran®), pipobroman (Amedel®, Vercyte®), triethylenemelamine (Hemel®, Hexylen®, Hexastat®), Demethyldopan®, Desmethyldopan®, triethylenethiophosphoramine, Temozolomide (Temodar®), thiotepa (Thioplex®), busulfan (Busilvex®, Myleran®), carmustine (BiCNU®), lomustine (CeeNU®), streptozocin (Zanosar®), and Dacarbazine (DTIC-Dome®). Additional example alkylating agents include, without limitation, Oxaliplatin (Eloxatin®); Melphalan (also known as L-PAM, L-sarcolysin, and phenylalanine mustard, Alkeran®); Altretamine (also known as hexamethylmelamine (HMM), Hexylen®); Carmustine (BiCNU®); Bendamustine (Treanda®); Busulfan (Busulfex® and Myleran®); Carboplatin (Paraplatin®); Temozolomide (Temodar® and Temodal®); Dactinomycin (also known as actinomycin-D, Cosmegen®); Lomustine (also known as CCNU, CeeNU®); Cisplatin (also known as CDDP, Platinol® and Platinol®-AQ); Chlorambucil (Leukeran®); Cyclophosphamide (Cytoxan® and Neosar®); Dacarbazine (also known as DTIC, DIC and imidazole carboxamide, DTIC-Dome®); Altretamine (also known as hexamethylmelamine (HMM), Hexylen®); Ifosfamide (Ifex®); Prednumustine; Procarbazine (Matulane®); Mechlorethamine (also known as nitrogen mustard, mustine and mechloroethamine hydrochloride, Mustargen®); Streptozocin (Zanosar®); Thiotepa (also known as thiophosphoamide, TESPA and TSPA, Thioplex®); Cyclophosphamide (Endoxan®, Cytoxan®, Neosar®, Procytox®, Revimmune); and Bendamustine HCl (Treanda®).

In some embodiments, the one or more additional therapeutic agents comprise a kinase inhibitor. In some embodiments, the kinase inhibitor comprises an inhibitor of EGFR, an inhibitor of c-Met, an inhibitor of HER2, an inhibitor of HER3, an inhibitor of HER4, an inhibitor of VEGFR, an inhibitor of AXL or a combination thereof. In certain embodiments, the kinase inhibitor is an inhibitor of EGFR. In particular embodiments, the kinase inhibitor is an inhibitor of c-Met. In some embodiments, the kinase inhibitor is an inhibitor of HER2. In certain embodiments, the kinase inhibitor is an inhibitor of HER3. In particular embodiments, the kinase inhibitor is an inhibitor of HER4. In some embodiments, the kinase inhibitor is an inhibitor of VEGFR. In certain embodiments, the kinase inhibitor is an inhibitor of or AXL.

In some embodiments, the kinase inhibitor comprises erlotinib, gefitinib, lapatinib, vandetanib, afatinib, osimertinib, lazertinib, mobocertinib, poziotinib, criotinib, cabozantinib, capmatinib, axitinib, lenvatinib, nintedanib, regorafenib, pazopanib, sorafenib, sunitinib or a combination thereof. In certain embodiments, the kinase inhibitor is erlotinib. In particular embodiments, the kinase inhibitor is gefitinib. In some embodiments, the kinase inhibitor is lapatinib. In certain embodiments, the kinase inhibitor is vandetanib. In some embodiments, the kinase inhibitor is afatinib. In some embodiments, the kinase inhibitor is osimertinib. In certain embodiments, the kinase inhibitor is lazertinib. In particular embodiments, the kinase inhibitor is poziotinib. In some embodiments, the kinase inhibitor is criotinib. In certain embodiments, the kinase inhibitor is cabozantinib. In some embodiments, the kinase inhibitor is capmatinib. In some embodiments, the kinase inhibitor is axitinib. In certain embodiments, the kinase inhibitor is lenvatinib. In some embodiments, the kinase inhibitor is nintedanib. In particular embodiments, the kinase inhibitor is regorafenib. In certain embodiments, the kinase inhibitor is pazopanib. In some embodiments, the kinase inhibitor is sorafenib. In particular embodiments, the kinase inhibitor is sunitinib. In some embodiments, the kinase inhibitor is mobocertinib.

In certain embodiments, the one or more prior anti-cancer therapies comprises carboplatin, paclitaxel, gemcitabine, cisplatin, vinorelbine, docetaxel, palbociclib, crizotinib, PD-(L)1 axis inhibitor, an inhibitor of EGFR, an inhibitor of c-Met, an inhibitor of HER2, an inhibitor of HER3, an inhibitor of HER4, an inhibitor of VEGFR, an inhibitor of AXL, erlotinib, gefitinib, lapatinib, vandetanib, afatinib, osimertinib, lazertinib, mobocertinib, poziotinib, criotinib, cabozantinib, capmatinib, axitinib, lenvatinib, nintedanib, regorafenib, pazopanib, sorafenib or sunitinib, or any combination thereof.

Anti-cancer therapies that may be administered in combination with the anti-EGFR/c-Met antibody (e.g., bispecific antibody) in the methods of the disclosure include any one or more of the chemotherapeutic drugs or other anti-cancer therapeutics known to those of skill in the art. Chemotherapeutic agents are chemical compounds useful in the treatment of cancer and include growth inhibitory agents or other cytotoxic agents and include alkylating agents, anti-metabolites, anti-microtubule inhibitors, topoisomerase inhibitors, receptor tyrosine kinase inhibitors, angiogenesis inhibitors and the like. Examples of chemotherapeutic agents include alkylating agents such as thiotepa and cyclosphosphamide (CYTOXAN®); alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaoramide and trimethylolomelamine; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, carminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-FU; folic acid analogues such as denopterin, methotrexate, pteropterin, trimetrexate; purine analogues such as fludarabine, 6-mercaptopurine, thiamiprine, thioguanine; pyrimidine analogues such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; amsacrine; bestrabucil; bisantrene; edatraxate; defofamine; demecolcine; diaziquone; elfornithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; PSK®; razoxane; sizofiran; spirogermanium; tenuazonic acid; triaziquone; 2,2′,2″-trichlorotriethylamine; urethan; vindesine; dacarbazine; mannomustine; mitobronitol; mitolactol; pipobroman; gacytosine; arabinoside (“Ara-C”); cyclophosphamide; thiotepa; members of taxoid or taxane family, such as paclitaxel (TAXOL®docetaxel (TAXOTERE®) and analogues thereof; chlorambucil; gemcitabine; 6-thioguanine; mercaptopurine; methotrexate; platinum analogues such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; xeloda; ibandronate; CPT-11; topoisomerase inhibitor RFS 2000; difluoromethylornithine (DMFO); retinoic acid; esperamicins; capecitabine; inhibitors of receptor tyrosine kinases and/or angiogenesis, including sorafenib (NEXAVAR®), sunitinib (SUTENT®), pazopanib (VOTRIENT™), toceranib (PALLADIA™), vandetanib (ZACTIMA™), cediranib (RECENTIN®), regorafenib (BAY 73-4506), axitinib (AG013736), lestaurtinib (CEP-701), erlotinib (TARCEVA®), gefitinib (IRESSA®), afatinib (BIBW 2992), lapatinib (TYKERB®), neratinib (HKI-272), and the like, and pharmaceutically acceptable salts, acids or derivatives of any of the above. Also included in this definition are anti-hormonal agents that act to regulate or inhibit hormone action on tumors such as anti-estrogens including for example tamoxifen, raloxifene, aromatase inhibiting 4(5)-imidazoles, 4-hydroxytamoxifen, trioxifene, keoxifene, LY 117018, onapristone, and toremifene (FARESTON®); and anti-androgens such as flutamide, nilutamide, bicalutamide, leuprolide, and goserelin; and pharmaceutically acceptable salts, acids or derivatives of any of the above. Other conventional cytotoxic chemical compounds as those disclosed in Wiemann et al., 1985, in Medical Oncology (Calabresi et al, eds.), Chapter 10, McMillan Publishing, are also applicable to the methods of the present invention.

In some embodiments, the anti-EGFR/c-Met antibody (e.g., bispecific antibody) and the one or more additional therapeutic agents (e.g., chemotherapeutic agents) are administered simultaneously. In other embodiments, the antibody and the one or more additional therapeutic agents are administered separately (e.g., sequentially).

For combination therapies, the one or more anti-cancer agents may be administered using recommended doses and dosages of the anti-cancer agent.

### Subjects

The terms “subject” and “patient” can be used interchangeably herein. “Patient in need thereof” or “subject in need thereof” refers to a mammalian subject, preferably human, diagnosed with or suspected of having a disease, to whom will be or has been administered a bispecific anti-EGFR anti-MET antibody according to a method of the invention. “Patient in need thereof” or “subject in need thereof” includes those subjects already with the undesired physiological change or disease well as those subjects prone to have the physiological change or disease.

In some embodiments, the subject is 18 years of age or older, e.g., 18 to less than 40 years of age, 18 to less than 45 years of age, 18 to less than 50 years of age, 18 to less than 55 years of age, 18 to less than 60 years of age, 18 to less than 65 years of age, 18 to less than 70 years of age, 18 to less than 75 years of age, 40 to less than 75 years of age, 45 to less than 75 years of age, 50 to less than 75 years of age, 55 to less than 75 years of age, 60 to less than 75 years of age, 65 to less than 75 years of age, 60 to less than 75 years of age, 40 years of age or older, 45 years of age or older, 50 years of age or older, 55 years of age or older, 60 years of age or older, 65 years of age or older, 70 years of age or older or 75 years of age or older.

In some embodiments, the subject is a child. In some embodiments, the subject is 18 years of age or younger, e.g., 0-18 years of age, 0-12 years of age, 0-16 years of age, 0-17 years of age, 2-12 years of age, 2-16 years of age, 2-17 years of age, 2-18 years of age, 3-12 years of age, 3-16 years of age, 3-17 years of age, 3-18 years of age, 4-12 years of age, 4-16 years of age, 4-17 years of age, 4-18 years of age, 6-12 years of age, 6-16 years of age, 6-17 years of age, 6-18 years of age, 9-12 years of age, 9-16 years of age, 9-17 years of age, 9-18 years of age, 12-16 years of age, 12-17 years of age or 12-18 years of age.

In some embodiments, the subject has been diagnosed with CRC (e.g., mCRC) for at least about 1 month, e.g., at least about: 2 months, 3 months, 4 months, 5 months, 6 months, 7 months, 8 months, 9 months, 10 months, 11 months, 1 year, 18 months, 2 years, 30 months, 3 years, 4 years, 5 years, 6 years, 7 years, 8 years, 9 years or 10 years. In particular embodiments, the subject is newly diagnosed with CRC (e.g., mCRC). In some embodiments, the CRC is adenocarcinoma.

In certain embodiments, the subject is treatment naïve.

In some embodiments, the subject has received one or more prior anti-cancer therapies. In certain embodiments, the one or more prior anti-cancer therapies comprises one or more chemotherapeutic agents, checkpoint inhibitors, targeted anti-cancer therapies or kinase inhibitors, or any combination thereof. In particular embodiments, the subject is relapsed or resistant to treatment with one or more prior anti-cancer therapies.

In some embodiments, the subject is resistant or has acquired resistance to an EGFR inhibitor. Exemplary EGFR inhibitors for which cancer may acquire resistance are anti-EGFR antibodies cetuximab (ERBITUX®), pantinumumab (VECTIBIX®), matuzumab, nimotuzumab, small molecule EGFR inhibitors erlotinib (TARCEVA®), gefitinib (IRESSA®), EKB-569 (pelitinib, irreversible EGFR TKI), pan-ErbB and other receptor tyrosine kinase inhibitors, lapatinib (EGFR and HER2 inhibitor), pelitinib (EGFR and HER2 inhibitor), vandetanib (ZD6474, ZACTIMA™, EGFR, VEGFR2 and RET TKI), PF00299804 (dacomitinib, irreversible pan-ErbB TKI), CI-1033 (irreversible pan-erbB TKI), afatinib (BIBW2992, irreversible pan-ErbB TKI), AV-412 (dual EGFR and ErbB2 inhibitor), EXEL-7647 (EGFR, ErbB2, GEVGR and EphB4 inhibitor), CO-1686 (irreversible mutant-selective EGFR TKI), AZD9291 (irreversible mutant-selective EGFR TKI), and HM-272 (neratinib, irreversible EGFR/ErbB2 inhibitor).

Various qualitative and/or quantitative methods may be used to determine if a subject is resistant, has developed or is susceptible to developing a resistance to treatment with an anti-cancer therapy. Symptoms that may be associated with resistance to an anti-cancer therapy include a decline or plateau of the well-being of the patient, an increase in the size of a tumor, arrested or slowed decline in growth of a tumor, and/or the spread of cancerous cells in the body from one location to other organs, tissues or cells. Re-establishment or worsening of various symptoms associated with cancer may also be an indication that a subject has developed or is susceptible to developing resistance to an anti-cancer therapy, such as anorexia, cognitive dysfunction, depression, dyspnea, fatigue, hormonal disturbances, neutropenia, pain, peripheral neuropathy, and sexual dysfunction. The symptoms associated with cancer may vary according to the type of cancer. For example, symptoms associated with cervical cancer may include abnormal bleeding, unusual heavy vaginal discharge, pelvic pain that is not related to the normal menstrual cycle, bladder pain or pain during urination, and bleeding between regular menstrual periods, after sexual intercourse, douching, or pelvic exam. Symptoms associated with lung cancer may include persistent cough, coughing up blood, shortness of breath, wheezing chest pain, loss of appetite, losing weight without trying and fatigue. Symptoms for liver cancer may include loss of appetite and weight, abdominal pain, especially in the upper right part of abdomen that may extend into the back and shoulder, nausea and vomiting, general weakness and fatigue, an enlarged liver, abdominal swelling (ascites), and a yellow discoloration of the skin and the whites of eyes (jaundice). One skilled in oncology may readily identify symptoms associated with a particular cancer type.

Exemplary PD-(L)1 axis inhibitors are antibodies that bind PD-1 such as nivolumab (OPDIVO®), pembrolimumab (KEYTRUDA®), sintilimab, cemiplimab (LIBTAYO®), tripolibamab, tislelizumab, spartalizumab, camrelizumab, dostralimab, genolimzumab or cetrelimab, or antibodies that bind PD-L1, such as PD-L1 antibodies are envafolimab, atezolizumab (TECENTRIQ®), durvalumab (IMFINZI®) and avelumab (BAVENCIO®).

Marketed antibodies may be purchased via authorized distributor or pharmacy. The amino acid sequences structures of the small molecules can be found from USAN and/or INN submissions by the companies of from CAS registry.

### Premedications

In some embodiments, the combination treatment, comprising an EGFR/c-Met antibody, also comprises one or more premedications. In some embodiments, the premedication comprises antihistamines, antipyretics, or glucocorticoids. In some embodiments, the premedication comprises an antihistamine. In some embodiments, the premedication comprises an antipyretic. In some embodiments, the premedication comprises a glucocorticoid. In some embodiments, the one or more premedications are administered as described in Table 1.

TABLE 1

| | | Route of |
|---|---|---|
| Medication | Dose | Administration |
| Antihistamine* | Diphenhydramine (25 to 50 mg) or | IV |
| | equivalent | Oral |
| Antipyretic* | Acetaminophen (650 to 1,000 mg) | IV |
| | | Oral |
| Glucocorticoid^{‡} | Dexamethasone (10 mg) or | IV |
| | Methylprednisolone (40 mg) or | Oral |
| | equivalent | |

*Required at all doses.
^{‡}Required at initial dose (Week 1, Days 1 and 2); optional for subsequent doses.

### Embodiments

1. A method of reducing occurrence or severity of infusion-related reactions (IRRs) in a subject treated with an anti-epidermal growth factor receptor (EGFR)/hepatocyte growth factor receptor (c-Met) antibody, comprising administering
   a) dexamethasone
   b) montelukast, or
   c) methotrexate
   to the subject.
2. The method of embodiment 1, wherein the antibody comprises:
   a) a first domain that specifically binds EGFR, comprising heavy chain complementarity determining region 1 (HCDR1), HCDR2, HCDR3, light chain complementarity determining region 1 (LCDR1), LCDR2 and LCDR3 amino acid sequences of SEQ ID NOs:1, 2, 3, 4, 5 and 6, respectively; and
   b) a second domain that specifically binds c-Met, comprising HCDR1, HCDR2, HCDR3, LCDR1, LCDR2 and LCDR3 amino acid sequences of SEQ ID NOs:7, 8, 9, 10, 11 and 12, respectively.
3. The method of embodiment 2, wherein the first domain comprises a heavy chain variable region (VH) of SEQ ID NO:13 and a light chain variable region (VL) of SEQ ID NO:14, and the second domain comprises a VH of SEQ ID NO:15 and a VL of SEQ ID NO:16.
4. The method of any one of embodiments 1-3, wherein the antibody is of the IgG1 isotype.
5. The method of any one of embodiments 1-4, wherein the antibody comprises a first heavy chain (HC1) of SEQ ID NO:17, a first light chain (LC1) of SEQ ID NO:18, a second heavy chain (HC2) of SEQ ID NO:19 and a second light chain (LC2) of SEQ ID NO:20.
6. The method of any one of embodiments 1-5, wherein the antibody is an isolated bispecific antibody.
7 The method of embodiment 6, wherein the bispecific antibody is amivantamab.
8. The method of any one of embodiments 1-7, wherein the antibody comprises a biantennary glycan structure with a fucose content of about 1% to about 15%.
9. The method of any one of embodiments 1-8, wherein the antibody is administered at a dose of about 700 mg to about 2,240 mg.
10. The method of embodiment 9, wherein the antibody is administered at a dose of about 700 mg, about 1,050 mg, about 1,400 mg, about 1,600 mg, or about 2,240 mg.
11. The method of embodiment 10, wherein the antibody is administered at a dose of about 1,400 mg.
12. The method of embodiment 10, wherein the antibody is administered at a dose of about 1,050 mg.
13. The method of embodiment 10, wherein the antibody is administered at a dose of about 700 mg.
13a. The method of embodiment 10, wherein the antibody is administered at a dose of about 1,600 mg.
13b. The method of embodiment 10, wherein the antibody is administered at a dose of about 2,240 mg.
14. The method of any one of embodiments 1-13, wherein the antibody is administered once a week or once every two weeks.
15. The method of embodiment 14, wherein the antibody is administered once weekly for the first 4 weeks and then every 2 weeks.
16. The method of any one of embodiments 1-15, wherein the antibody is administered on a 28-day cycle.
17. The method of any one of embodiments 1-16, wherein the antibody is administered as a monotherapy.
18. The method of any one of embodiments 1-16, wherein the subject treated with the anti-EGFR/c-Met antibody is further administered one or more chemotherapeutic agents.
19. The method of embodiment 18, wherein the one or more chemotherapeutic agents comprise a tyrosine kinase inhibitor (TKI).
20. The method of embodiment 18, wherein the one or more chemotherapeutic agents comprise Lazertinib, osimertinib, capmatinib, or mobocertinib.
20a. The method of Embodiment 20, wherein the one or more chemotherapeutic agents comprise Lazertinib.
20b. The method of Embodiment 20, wherein Lazertinib is administered at a dose of 240 mg.
20c. The method of Embodiment 20, wherein the one or more chemotherapeutic agents comprise osimertinib.
20d. The method of Embodiment 20, wherein the one or more chemotherapeutic agents comprise mobocertinib.
20e. The method of Embodiment 20, wherein the one or more chemotherapeutic agents comprise capmatinib.
21. The method of any one of embodiments 1-20, wherein cancer is a non-small cell lung cancer (NSCLC), an epithelial cell cancer, a breast cancer, an ovarian cancer, a lung cancer, a squamous cell lung cancer, a lung adenocarcinoma, a small cell lung cancer, a colorectal cancer, an anal cancer, a prostate cancer, a kidney cancer, a bladder cancer, a head and neck cancer, a pharynx cancer, a cancer of the nose, a pancreatic cancer, a skin cancer, an oral cancer, a cancer of the tongue, an esophageal cancer, a vaginal cancer, a cervical cancer, a cancer of the spleen, a testicular cancer, a gastric cancer, a cancer of the thymus, a colon cancer, a thyroid cancer, a liver cancer, a hepatocellular carcinoma (HCC) or sporadic or hereditary papillary renal cell carcinoma (PRCC), head and neck squamous cell carcinoma (HNSCC).
22. The method embodiment 21, wherein the cancer is NSCLC.
22a. The method of any one of embodiments 1, 21, or 22, wherein cancer is associated with EGFR exon 19 deletion.
22b. The method of any one of embodiments 1, 21, or 22, wherein cancer is associated with L858R mutation.
23. The method of any one of embodiments 1-22, wherein the subject has been diagnosed with left-sided mCRC.
24. The method of any one of embodiments 1-22, wherein the subject has been diagnosed with right-sided mCRC.
25. The method of any one of embodiments 1-24, wherein the subject is anti-EGFR therapy naïve.
26. The method of any one of embodiments 1-24, wherein the subject has received prior anti-EGFR therapy.
27. The method of any one of embodiments 1-25, wherein the subject is treatment naïve.
28. The method of any one of embodiments 1-26, wherein the subject is relapsed or resistant to treatment with one or more prior anti-cancer therapies.
28a. The method of Embodiment 28, wherein the prior anti-cancer therapy is osimertinib.
28b. The method of Embodiment 28, wherein the prior anti-cancer therapy is platinum chemotherapy.
29. The method of any one of embodiments 1-28, wherein the subject is 18 years of age or older.
30. The method of any one of embodiments 1-29, further comprising administering a premedication with one or more of antihistamines, antipyretics, or glucocorticoids.
30a. The method of Embodiment 30, wherein the premedication comprises a antihistamine.
30b. The method of Embodiment 30, wherein the premedication comprises a antipyretic.
30c. The method of Embodiment 30, wherein the premedication comprises a glucocorticoid.
31. The method of any one of embodiments 1-30, wherein methotrexate is administered between 5 days to 1 day prior to the administration of the anti-EGFR/c-Met antibody.
32. The method of embodiment 31, wherein methotrexate is administered at a dose of 25 mg.
33. The method of any one of embodiments 1-30, wherein montelukast is administered daily starting 4 days prior to the administration of the anti-EGFR/c-Met antibody.
34. The method of embodiment 33, wherein montelukast is administered 5 times.
35. The method of embodiment 33, wherein montelukast is administered at a dose 10 mg.
36. The method of any of embodiments 30-35, further comprising administration of IV dexamethasone on the first and second days of administering the anti-EGFR/c-Met antibody, wherein the administration of dexamethasone is 45-60 minutes prior to the administration of the anti-EGFR/c-Met antibody.
37. The method of embodiment 36, wherein IV dexamethasone is administered at a dose of 10 mg.
38. The method of any one of embodiments 1-30, wherein oral dexamethasone is administered 1 day prior to the administration of the anti-EGFR/c-Met antibody.
39. The method of embodiment 38, wherein oral dexamethasone is administered at a total daily dose of 8 mg.
40. The method of any of embodiments 38-39, further comprising administration of IV dexamethasone on the first and second days of administering the anti-EGFR/c-Met antibody, wherein IV dexamethasone is administered at a dose between 10-20 mg.
41. The method of embodiment 40, wherein IV dexamethasone is administered at a dose of 20 mg on the first day and 10 mg on the second day.
42. The method of any of embodiments 1-41, further comprising administration of a premedication with one or more of antihistamines, antipyretics, or glucocorticoids.
43. The method of embodiment 42, wherein the premedication comprises diphenhydramine.
44. The method of embodiment 43, wherein the diphenhydramine is administered at a dose of 25 to 50 mg.
45. The method of embodiment 42, wherein the premedication comprises acetaminophen.
46. The method of embodiment 45, wherein the acetaminophen is administered at a dose of 650 to 1,000 mg.

### Example 1. Methotrexate, Montelukast, or Dexamethasone for the Reduction of Amivantamab Infusion-Related Reactions

### Objectives and Endpoints

The primary objective is to assess the prophylaxis efficiency of methotrexate, montelukast, or dexamethasone prior to lazertinib, and IV amivantamab infusion to decrease incidence and/or severity of IRRs.

### Overall Design

This is a proof-of-concept, open-label, multicenter study in participants with EGFR exon 19 deletion or L858R mutated NSCLC who have progressed on or after prior osimertinib and on or after platinum chemotherapy, who may benefit from IV amivantamab+lazertinib combination therapy. In study, participants will receive standard prophylaxis with antihistamine, antipyretic, and glucocorticoid.

There are three cohorts in the study.
Cohort A: Participants will be administered oral dexamethasone (4 mg) twice a day (8 mg total daily dose) on Day −1 (Cycle 1) prior to lazertinib and IV amivantamab combination therapy in addition to all other standard premedication.
Cohort B: Participants will be administered oral montelukast (10 mg) in the morning on Days −4, −3, −2, −1, and C1D1 (5 doses total) prior to lazertinib and IV amivantamab combination therapy.
Cohort C: Participants will be administered a single dose of 25 mg subcutaneous (SC) methotrexate on any day between Days −7 and Day −3 (Cycle 1) prior to lazertinib and IV amivantamab combination therapy.

Study treatments will be administered in addition to all other standard premedications.

### Study Population

Screening for eligible participants will be performed within 28 days before administration of lazertinib and IV amivantamab. The inclusion and exclusion criteria for enrolling participants in this study are described below.

### Inclusion Criteria:

≥18 years of age (or the legal age of consent in the jurisdiction in which the study is taking place) at the time of informed consent.

Participant must have advanced or metastatic NSCLC.

Progressed on or after prior treatment with osimertinib and platinum-based chemotherapy. Prior use of first-or-second generation EGFR TKI is allowed if administered prior to osimertinib.

Previously identified EGFR-mutated NSCLC (EGFR Exon19 deletion or L858R) (identified locally in a Clinical Laboratory Improvement Amendments [CLIA]-certified laboratory [or equivalent])

ECOG performance status grade of 0 or 1 (Eastern Cooperative Oncology Group, Robert Comis M. D, Group Chair (Oken, 1982)).

Subject must have organ and bone marrow function as follows:

a) Hemoglobin ≥9 g/dL

b) ANC ≥1.5×109/L

c) Platelets ≥75×10 9/L

d) AST and ALT ≤3×ULN

e) Total bilirubin ≤1.5×ULN; subjects with Gilbert's syndrome can enroll if

conjugated bilirubin is within normal limits

f) Have an estimated glomerular filtration rate (eGFR), based on the Modified Diet in Renal Disease (MDRD) 4-variable formula (see Attachment 5), of >30 mL/min.

### Treatment Groups and Duration

EGFR exon 19 or L858R mutated advanced NSCLC with disease progression on or after osimertinib and doublet platinum chemotherapy and will proceed with the study treatment of IV amivantamab+lazertinib as determined by investigator.

Description of Study Treatments.

Methotrexate

Methotrexate (MTX) [N-[4-[[(2,4-diamino-6-pteridinyl)methyl]methylamino]benzoyl]-Lglutamic acid], is an FDA-approved folic acid antagonist indicated for the treatment of rheumatoid arthritis. MTX is an anti-metabolite, commonly used in chemotherapy and immunosuppressant in auto-immune diseases.

Methotrexate inhibits dihydrofolic acid reductase, interfering with DNA synthesis, repair, and cellular replication. Actively proliferating tissues such as malignant cells, bone marrow, fetal cells, buccal and intestinal mucosa, and cells of the urinary bladder are in general more sensitive to this effect of methotrexate.

The mechanism of action in rheumatoid arthritis is unknown; it may affect immune function by inhibiting the enzyme aminoimidazole carboxamide riboside transformylase, leading to hindrance in adenosine and guanine metabolism, adenosine accumulation; and due to anti-inflammatory action of adenosine, leads to repression of T-cell activation, down-regulation of B-cells, increasing activated CD-95 T cells sensitivity; and repression of methyltransferase activity, inhibition of the binding of beta-1 interleukin to its cell surface receptor.

Montelukast

Montelukast is an oral, FDA-approved for the treatment of chronic asthma and prophylaxis and the prevention of exercise-induced bronchoconstriction. It is also approved for the relief of symptoms of both seasonal and perennial allergic rhinitis.

Montelukast inhibits the mast cell mediated release of leukotrienes and may be used to reduce inflammation and bronchoconstriction.

Montelukast is a highly selective leukotriene receptor antagonist that binds with high affinity to leukotrienes, which are excreted by various types of cells, including mast cells, and are involved in the inflammatory process that may cause the signs and symptoms of asthma and allergic rhinitis. Leukotriene receptors are found in airway cells, such as macrophages and smooth muscle cells. When bound to leukotriene receptors, montelukast inhibits leukotriene physiologic effects (such as airway edema, smooth muscle contraction, and impairment of normal cellular activity). This serves as the rationale for montelukast to potentially reduce the symptomatology (e.g., dyspnea) associated with amivantamab IRRs.

Dexamethasone

Dexamethasone is a synthetic adrenocortical steroid available in oral and IV formulations FDA-approved for allergic states. IV dexamethasone (10 mg) is a standard premedication administered prior to all patients receiving IV amivantamab. This study will investigate the role of an enhanced steroid pre-loading to reduce the incidence of IV amivantamab IRRs.

The study treatments, dexamethasone, montelukast, and methotrexate, will be investigated prior to administration of the background anti-cancer combination regimen of lazertinib plus IV amivantamab to assess the incidence of IRRs.

Study treatments will be administered in addition to all other standard premedication. Standard premedication with antihistamines, antipyretics, and/or glucocorticoids is recommended, including premedication with dexamethasone 10 mg IV required at IV amivantamab initial dose (Week 1, Days 1 and 2).

Summary of Schedule of Activities is shown in Table 2. Suggested order of administration are shown in Tables 3-5.

TABLE 2
Summary of Schedule of Activities.

| | Study Period | | | | | | | | | | | | | | | | |
|---|---|---|---|---|---|---|---|---|---|---|---|---|---|---|---|---|---|
| | | | | | | | | | | | | | | | | End of | |
| | | | | | | | | | | | | | | | | Study | |
| | | | | | | | | | | | | | | | | Up to 30 | |
| | | | | | | | | | | | | | | | | days after | |
| | | | | | | | | | | | | | | | | Cycle 3 | |
| | | Prophylaxis | | | | | Treatment (28 days/cycle) | | | | | | | | | or last | |
| | Screening | Administration | | | | | Cycle 1 | | | | | Cycle 2 | | Cycle 3 | | dose | |
| | Cycle Day | | | | | | | | | | | | | | | | |
| | | −7 | | | | | | | | | | | | | | | |
| | | to −3 of | | | | | | | | | | | | | | | |
| | | C1D1 | −4 | −3 | −2 | −1 | 1 | 2 | 8 | 15 | 22 | 1 | 15 | 1 | 15 | | |
| | Visit Window (Days) | | | | | | | | | | | | | | | | |
| | −28 | −7 | | | | | | | | | | | | | | | |
| | to −6 | to −3 | | | | | — | | ±1 | ±2 | | ±1 | | ±3 | | 0 | Notes |
| STUDY TREATMENT ADMINISTRATION by Cohort | | | | | | | | | | | | | | | | | |
| A: Oral | | | | | | X | | | | | | | | | | | Cohort A: Dexamethasone |
| Dexamethasone | | | | | | | | | | | | | | | | | (4 mg) PO BID (8 mg |
| B: | | | X | X | X | X | X | | | | | | | | | | total) Cycle 1 Day (−1). |
| Montelukast | | | | | | | | | | | | | | | | | Cohort B: montelukast (10 |
| C: | | X | | | | | | | | | | | | | | | mg) PO QD for 5 days |
| Methotrexate | | | | | | | | | | | | | | | | | ending on C1D1. |
| | | | | | | | | | | | | | | | | | Cohort C: methotrexate is |
| | | | | | | | | | | | | | | | | | administered only once |
| | | | | | | | | | | | | | | | | | anytime from −7 to −3 of |
| | | | | | | | | | | | | | | | | | C1D1 |
| BACKGROUND ANTI-CANCER TREATMENT ADMINISTRATION | | | | | | | | | | | | | | | | | |
| Lazertinib | | | | | | | Once daily oral administration | | | | | | | | | | |
| IV | | | | | | | X | X | X | X | X | X | X | X | X | | |
| amivantamab | | | | | | | | | | | | | | | | | |
| PRE-INFUSION MEDICATIONS | | | | | | | | | | | | | | | | | |
| IV | | | | | | | X | X | | | | | | | | | Dexamethasone (10 mg) |
| Dexamethasone | | | | | | | | | | | | | | | | | IV 45-60 minutes prior to |
| (10 mg) | | | | | | | | | | | | | | | | | IV amivantamab (C1D1 |
| | | | | | | | | | | | | | | | | | and C1D2). |
| Diphenhydramine | | | | | | | X | X | | | | X | X | X | X | | Diphenhydramine (25 to |
| | | | | | | | | | | | | | | | | | 50 mg) or equivalent IV |
| | | | | | | | | | | | | | | | | | 15-30 minutes prior to IV |
| | | | | | | | | | | | | | | | | | amivantamab (all cycles, |
| | | | | | | | | | | | | | | | | | all cohorts) or |
| | | | | | | | | | | | | | | | | | diphenhydramine (25 to |
| | | | | | | | | | | | | | | | | | 50 mg) or equivalent PO |
| | | | | | | | | | | | | | | | | | 30-60 minutes prior to IV |
| | | | | | | | | | | | | | | | | | amivantamab (all cycles, |
| | | | | | | | | | | | | | | | | | all cohorts). |
| Paracetamol | | | | | | | X | X | | | | X | X | X | X | | Paracetamol |
| or | | | | | | | | | | | | | | | | | (acetaminophen 650 to |
| acetaminophen | | | | | | | | | | | | | | | | | 1,000 mg) or |
| | | | | | | | | | | | | | | | | | equivalent IV 15-30 |
| | | | | | | | | | | | | | | | | | minutes prior to IV |
| | | | | | | | | | | | | | | | | | amivantamab (all cycles, |
| | | | | | | | | | | | | | | | | | all cohorts). |

TABLE 3
Suggested Order of Administration in Cohort A.

| | | Route of | |
|---|---|---|---|
| Medication | Dose | Administration | Recommended Dosing Window |
| Study | Dexamethasone 4 mg | Oral | 1 day before the first amivantamab infusion on |
| Treatment | twice a day (8 mg total | | Day −1 (Cycle 1) |
| | daily dose) | | |
| Glucocorticoid | Days 1 and 2 of Cycle 1: | IV | Start 60-90 minutes before amivantamab infusion |
| | Dexamethasone 10 mg | | |
| Antipyretic | Paramcetemol | IV or oral | Start 15-30 minutes before amivantamab infusion |
| | (acetaminophen) 650 to | | |
| | 1000 mg (or equivalent)* | | |
| Antihistamine | Diphenhydramine 25 mg | IV or oral | |
| | or equivalent* | | |
| Background | Lazertinib 240 mg | Oral | lazertinib should be dosed no more than 15 minutes |
| anti-cancer | | | before the start of each amivantamab infusion. Lazertinib |
| treatment | | | should be taken at approximately the same time each day, |
| | | | approximately 24 hours apart. if possible |
| Background | Amivantamab | IV | 1050 mg (for patients <80 kg) or 1400 mg (for |
| anti-cancer | | | patients ≥80 kg) once weekly for 4 weeks. then |
| treatment | | | every 2 weeks therafter |
| | | | Initial dose of IV amivantamab (Cycle 1, Days 1 and 2), |
| | | | is administered as a split dose over 2 days (eg. Cycle 1 |
| | | | Day 1 [350 mg] and Cycle 1 Day 2 [remainder of dose]). |

*required premedication for amivantamab

TABLE 4
Suggested Order of Administration in Cohort B.

| | | Route of | |
|---|---|---|---|
| Medication | Dose | Administration | Recommended Dosing Window |
| Study | Montelukast 10 mg | Oral | Montelukast taken in the morning on |
| Treatment | | | days −4, −3, −2, −1 and C1D1 (5 doses total). |
| | | | Montelukast should be taken at approximately the |
| | | | same time each day, approximately 24 hours apart. |
| | | | if possible |
| Glucocorticoid | Days 1 and 2 of Cycle 1: | IV | Start 60-90 minutes before amivantamab infusion |
| | Dexamethasone 10 mg | | |
| Antipyretic | Paracetamol | IV or oral | Start 15-30 minutes before amivantamab infusion |
| | (acetaminophen) 650 to | | |
| | 1000 mg (or equivalent)* | | |
| Antihistamine | Diphenhydramine 25 mg | IV or oral | |
| | or equivalent* | | |
| Background | Lazertinib 240 mg | Oral | lazertinib should be dosed no more than 15 minutes |
| anti-cancer | | | before the start of each amivantamab infusion. |
| treatment | | | Lazertinib should be taken at approximately the |
| | | | same time each day, approximately 24 hours apart. |
| | | | if possible |
| Background | Amivantamab | IV | 1050 mag (for patients <80 kg) or 1400 mg (for |
| anti-cancer | | | patients ≥80 kg) once weekly for 4 weeks, then |
| treatment | | | every 2 weeks thereafter. |
| | | | Initial dose of IV amivantamab (Cycle 1, Days 1 and |
| | | | 2), is administered as a split dose over 2 days (eg. |
| | | | Cycle 1 Day 1 [350 mg] and Cycle 1 Day 2 |
| | | | [remainder of dose]). |

*required premedication for amivantamab

TABLE 5
Suggested Order of Administration in Cohort C.

| | | Route of | |
|---|---|---|---|
| Medication | Dose | Administration | Recommended Dosing Window |
| Study | Methotrexate 25 mg | SC | Single dose on any day between |
| treatment | | | Days −7 and Day −3 (Cycle 1) prior to Lazertinib and IV |
| | | | amivantamab combination therapy. |
| Glucocorticoid | Days 1 and 2 of Cycle 1: | IV | Start 60-90 minutes before amivantamab infusion |
| | Dexamethasone 10 mg | | |
| Antipyretic | Paracetamol | IV or oral | Start 13-30 minutes before amivantamab infusion |
| | (acetaminophen) 650650 to | | |
| | 1000 mg (or equivalent)* | | |
| Antihistamine | Diphenhydramine 25 mg | IV | |
| | or equivalent* | | |
| Background | Lazertinib 240 mg | Oral | lazertinib should be dosed no more than 15 minutes |
| anti-cancer | | | before the start of each amivantamab infusion. Lazertinib |
| treatment | | | should be taken at approximately the same time each day. |
| | | | approximately 24 hours apart, if possible |
| Background | Amivantamab | IV | 1050 mg (for patients <80 kg) or 1400 mg (for |
| anti-cancer | | | patients ≥80 kg) once weekly for 4 weeks, then |
| treatment | | | every 2 weeks thereafter. |
| | | | Initial dose of IV amivantamab (Cycle 1, Days 1 and 2), |
| | | | is administered as a split dose over 2 days (eg. Cycle 1 |
| | | | Day 1 [350 mg] and Cycle 1 Day 2 [remainder of dose]). |

*required premedication for amivantamab

### Dose Modifications.

Doses for IV amivantamab or Lazertinib may be modified, if determined by qualified study site personnel. If the experienced toxicity is felt to be attributable to either IV amivantamab or lazertinib, then the dose of the responsible agent should be preferentially modified.

In some embodiments, when dose modification is required, modification may occur as listed below (Table 6).

TABLE 6
Dose Modifications for Lazertinib and IV amivantamab.

| Combination | Dose of | Dose of IV amivantamab (mg) IV |
|---|---|---|
| dosing level | lazertinib (mg) | (dose <80 kg/dose ≥88 kg) |
| 1 | 240 | 1050/1400 |
| 2 | 160 | 1050/1400 |
| 3 | 160 | 700/1050 |
| 4 | 80 | 700/1050 |
| 5 | Discontinue | 1050/1400 |

In some embodiments, if holding of lazertinib or IV amivantamab is considered clinically indicated, the decision to hold lazertinib or IV amivantamab dosing may be guided by the experienced toxicity and the likelihood of either treatment contributing to the toxicity, based upon the safety profile of both treatments.

In some embodiments, if both treatments are held, and are to be restarted, lazertinib may be restarted first and dosed for approximately 7 days prior to the next infusion of IV amivantamab.

### Treatment of Infusion-Related Reactions

Participants who experience early symptoms of IRRs, manifesting as, but not limited to, chills, nausea, dyspnea, flushing, chest discomfort, vomiting, or any other symptoms during the time of the infusion, may have their infusions interrupted, if indicated, and the symptoms managed according to the recommendations provided in Table 7. With the initial dose of IV amivantamab (Cycle 1, Days 1 and 2), interruption of the infusion may be considered even with mild symptoms to prevent more severe manifestations of IRR.

TABLE 7
Management of Infusion-related Reactions

| | | Premedication at |
|---|---|---|
| Toxicity Grade* | Treatment | subsequent dosing |
| Grade 1 or 2 | Interrupt IV amivantamab infusion if IRR is | Antihistamine, antipyretic, |
| Grade 1: | suspected and monitor patient until reaction | and glucocorticoid |
| Mild reaction | symptoms resolve. | |
| Grade 2: | Resume the infusion at 50% of the infusion | Antihistamine, antipyretic, |
| Mild to moderate reaction: | rate at which the reaction occurred. | and glucocorticoid |
| therapy or infusion interrupted | If there are no additional symptoms after | Consider meperidine if |
| but responds promptly to | 30 minutes, the infusion rate may be | subject experiences chills |
| symptomatic treatment | escalated. | and rigors. |
| | Include corticosteroid with premedications | |
| | for subsequent dose. | |
| Grade 3 | Interrupt IV amivantamab infusion and | Based on severity of |
| Severe reaction | administer supportive care medications. | symptoms, consider |
| Grade 3: prolonged (ie, not | Monitor patient until reaction symptoms | permanent discontinuation |
| rapidly responsive to | resolve. | of IV amivantamab. |
| symptomatic medication | Resume the infusion at 50% of the infusion | Discussion with Sponsor |
| and/or brief interruption of | rate at which the reaction occured. | required before continuing |
| infusion); recurrence of | If there are no additional symptoms after | with subsequent dosing. |
| symptoms following initial | 30 minutes, the infusion rate may be | |
| improvement; hospitalization | escalated. | |
| indicated for other clinical | Include corticosteroid with premedications | |
| sequelae (eg, renal | for subsequent dose. For recurrent Grade 3, | |
| impairment, pulmonary | permanently discontinue IV amivantamab. | |
| infiltrates) | | |
| Grade 4: life-threatening: | Permanently discontinue IV amivantamab | |
| pressor or ventilator support | | |
| indicated | | |
| General | Prophylactic medications (after initial event) | |
| | may be used as described in the IV amivantamab | |
| | product label. Appropriate personnel and | |
| | appropriate resuscitation equipment should be | |
| | available in or near the infusion room and a | |
| | physician should be readily available during the | |
| | infusion of IV amivantamab | |

*Per NCI Common Terminology Criteria for Adverse Events (CTCAE) Version 4.03

### Infusion-Related Reactions

Infusion-related reactions have been observed during treatment with various monoclonal anti-EGFR antibodies. Infusion-related reactions have been also observed during treatment with bispecific anti-EGFR/anti-MET antibodies. The severity of infusion reactions has been variable.

Signs and symptoms of IRR may include chills, dyspnea, flushing, nausea, chest discomfort, vomiting, tachycardia, hypotension, and fever.

### Concomitant Therapy Considerations.

Methotrexate Cohort.

Contraindicated therapies must be discontinued at least 3 weeks or 5 half-lives, whichever is shorter, before the first dose of study treatment (dexamethasone, montelukast or methotrexate).

Methotrexate is contraindicated in pregnancy, alcoholism or liver disease, immunodeficiency syndromes, preexisting blood dyscrasias, and hypersensitivity to methotrexate (MTX).

NSAIDs, salicylates, TMP, penicillin, warfarin, valproate, proton pump inhibitors, cyclosporin, cisplatin increases the risk of MTX toxicity in the blood; aminoglycosides, neomycin, probenecid reduces the absorption of MTX.

Drug Interactions:

Aspirin, NSAIDs, and steroids: concomitant use may elevate and prolong serum methotrexate levels and cause increased toxicity.

Proton pump inhibitors: concomitant use may elevate and prolong serum methotrexate levels and cause increased toxicity.

Oral antibiotics

Hepatotoxins

Theophylline

Folic acid and antifolates

Mercaptopurine

Nitrous oxide

As methotrexate is highly plasma protein bound, any drug that displaces methotrexate from proteins can increase its blood levels.

Montelukast Cohort

Montelukast is contraindicated in patients with a history of hypersensitivity to the drug or its components. For patients with phenylketonuria (PKU), caution should be exercised with phenylalanine-containing formulations.

Lazertinib and IV Amivantamab

The following concomitant medications and therapies are prohibited during the study:

Any chemotherapy, anti-cancer therapy (other than lazertinib and IV amivantamab), or experimental therapy.

Concomitant use of medications, herbal supplements and/or ingestions of foods with known potent inducer or inhibitory effects on CYP3A4/A5 activity should be avoided. Drugs that are potent inhibitors of CYP3A4 activity must have been discontinued for an appropriate period before administration of lazertinib.

Lazertinib is an inhibitor of P-glycoprotein (P-gp), multi-drug resistance protein 4 (MRP4), Breast Cancer Resistance Protein (BCRP), and Organic Cation Transporter 1 (OCT1). Therefore, concomitant administration of medications, herbal supplements and/or ingestions of foods with that are substrates of P-gp, MRP4, BCRP, or OCT1 are not recommended.

Lazertinib has the potential for reversible and time dependent inhibition of CYP3A4. Concomitant use of CYP34A substrate drugs must be discontinued before administration of lazertinib.

### Efficacy Evaluation

The primary objective is to assess the prophylaxis efficiency of methotrexate, montelukast, or dexamethasone prior to lazertinib and IV amivantamab infusion to reduce first-dose IRRs. The primary endpoint is rate of IRRs occurring on Cycle 1 Day 1 following administration of Lazertinib and IV amivantamab combination therapy. Signs and symptoms of IRR may include: chills, dyspnea, flushing, nausea, chest discomfort, vomiting, tachycardia, hypotension, and/or fever.

During the IV amivantamab infusion, subjects should be clinically monitored at regular intervals (including an assessment prior to the start of infusion). Vital signs should be measured within 30 minutes prior to IV amivantamab administration. On Cycle 1 Day 1, vital signs should also be measured 2 hours±15 min after the IV amivantamab administration. The monitoring should include pulse/heart rate, blood pressure, temperature, respiratory rate, and oxygen saturation measurements.

Secondary endpoints are rates and severity of individual AEs associated with IRR signs and symptoms (chills, dyspnea, flushing, nausea, chest discomfort, vomiting, tachycardia, hypotension, fever) as defined by the NCI CTCAE Criteria, Version 5.0 during Cycle 1 Day 1, rates and severity of these AEs on subsequent administrations up to 3 months, severity of infusion-related reactions, incidence of other AEs, and median duration of infusion time for pre-amivantamab infusion medications, IV amivantamab infusion, post-amivantamab infusion medications, investigator assessed tumor response and duration of response.

this study is not a measure of the background anti-cancer therapy (IV amivantamab and lazertinib), however, assessment of responses for solid tumors will be performed according to RECIST v1.1 criteria (European Journal of Cancer 45 (2009) 228-247).

### Safety Evaluations

The safety of the study treatments (methotrexate, montelukast, and dexamethasone) will be assessed by physical examinations, clinical laboratory tests, vital signs, electrocardiograms, monitoring of adverse events (AEs), and concomitant medication usage.

The safety of the background anti-cancer therapy (IV amivantamab and lazertinib) will be assessed by physical examinations, clinical laboratory tests, vital signs, electrocardiograms, monitoring of adverse events (AEs), and concomitant medication usage.

There will be active safety surveillance during the first three months of treatment and until end of study and passive safety surveillance where sites may follow the subjects for disease assessment and safety per the local practice during the open-label

### Statistical Methods

The primary hypothesis of this study is that through prophylactic treatment with dexamethasone, methotrexate or montelukast, the incidence of IV amivantamab IRRs on Cycle 1 Day 1 will be reduced.

In this study, the null hypothesis that the true IRR rate is 0.67 or higher will be tested against a one-sided alternative for each prophylaxis cohort.

For sample size determination, Simon's two-stage design (Simon, 1989) will be used separately for each cohort. Each cohort can be expanded.

The primary endpoint is the Rate of IRRs occurring on Cycle 1 Day 1 following administration of lazertinib and IV amivantamab combination therapy. Primary analysis of IRR rate will be performed after the last subject receives the first infusion or at the end of study, whichever comes first. Treated subject population will be used for primary analysis. IRR rate along with 95% confidence interval will be estimated for the cohorts.

Secondary endpoints are rates and severity of individual AE signs and symptoms of IRR (chills, dyspnea, flushing, nausea, chest discomfort, vomiting, tachycardia, hypotension, fever) occurring on Cycle 1 Day 1, and on subsequent administrations of IV amivantamab for up to 3 months, the severity of infusion-related reactions, incidence of other adverse events, rates of IRR following subsequent administrations, median duration of infusion time for pre-amivantamab infusion medications, IV amivantamab infusion, and post-amivantamab infusion medications and investigator assessed ORR and duration of response.

The secondary endpoint of ORR with confirmed best overall responses will be performed approximately 12 weeks after the last subject receives the first infusion or at the end of study, whichever comes first. ORR is defined as the proportion of subjects who achieve either a complete (CR) or partial response (PR) as defined by investigator assessment using RECIST v1.1. Observed ORR along with their two-sided exact 95% CIs will be presented for each cohort as appropriate.

Duration of response will be estimated using the Kaplan-Meier method and calculated as time from initial response of CR or PR to progressive disease (PD) or death due to underlying disease, whichever comes first, only for subjects who achieve CR or PR.

What is claimed is:

1. A method of reducing occurrence or severity of infusion-related reactions (IRRs) in a subject treated with an anti-epidermal growth factor receptor (EGFR)/hepatocyte growth factor receptor (c-Met) antibody, comprising administering
a) dexamethasone,
b) montelukast, or
c) methotrexate
to the subject.

2. The method of claim 1, wherein the antibody comprises:
a) a first domain that specifically binds EGFR, comprising heavy chain complementarity determining region 1 (HCDR1), HCDR2, HCDR3, light chain complementarity determining region 1 (LCDR1), LCDR2 and LCDR3 amino acid sequences of SEQ ID NOs: 1, 2, 3, 4, 5 and 6, respectively; and
b) a second domain that specifically binds c-Met, comprising HCDR1, HCDR2, HCDR3, LCDR1, LCDR2 and LCDR3 amino acid sequences of SEQ ID NOs: 7, 8, 9, 10, 11 and 12, respectively.

3. The method of claim 1, wherein the first domain comprises a heavy chain variable region (VH) of SEQ ID NO:13 and a light chain variable region (VL) of SEQ ID NO:14, and the second domain comprises a VH of SEQ ID NO:15 and a VL of SEQ ID NO:16.

4. The method of claim 1, wherein the antibody is of the IgG1 isotype.

5. The method of claim 1, wherein the antibody comprises a first heavy chain (HC1) of SEQ ID NO:17, a first light chain (LC1) of SEQ ID NO:18, a second heavy chain (HC2) of SEQ ID NO:19 and a second light chain (LC2) of SEQ ID NO:20.

6. The method of claim 1, wherein the antibody is an isolated bispecific antibody.

7. The method of claim 1, wherein the bispecific antibody is amivantamab.

8. The method of claim 1, wherein the antibody is administered at a dose of about 1,050 mg, about 1,400 mg, about 1,600 mg, or about 2,240 mg.

9. The method of claim 8, wherein the antibody is administered at a dose of about 1,400 mg.

10. The method of claim 8, wherein the antibody is administered at a dose of about 1,050 mg.

11. The method of claim 8, wherein the antibody is administered at a dose of about 1,600 mg.

12. The method of claim 8, wherein the antibody is administered at a dose of about 2,240 mg.

13. The method of claim 1, wherein the antibody is administered once a week or once every two weeks.

14. The method of claim 13, wherein the antibody is administered once weekly for the first 4 weeks and then every 2 weeks.

15. The method of claim 1, wherein the antibody is administered as a monotherapy.

16. The method of claim 1, wherein the subject treated with the anti-EGFR/c-Met antibody is further administered one or more chemotherapeutic agents.

17. The method of claim 16, wherein the one or more chemotherapeutic agents comprise a tyrosine kinase inhibitor (TKI).

18. The method of claim 17, wherein the one or more chemotherapeutic agents comprise lazertinib.

19. The method of claim 17, wherein the one or more chemotherapeutic agents comprise osimertinib.

20. The method of claim 1, wherein methotrexate is administered between 7 days to 3 days prior to the administration of the anti-EGFR/c-Met antibody.

21. The method of claim 20, wherein methotrexate is administered at a dose of 25 mg.

22. The method of claim 1, wherein montelukast is administered daily starting 4 days prior to the administration of the anti-EGFR/c-Met antibody.

23. The method of claim 22, wherein montelukast is administered 5 times.

24. The method of claim 23, wherein montelukast is administered at a dose 10 mg.

25. The method of claim 20 or 22, further comprising administration of IV dexamethasone on the first and second days of administering the anti-EGFR/c-Met antibody, wherein the administration of dexamethasone is 45-60 minutes prior to the administration of the anti-EGFR/c-Met antibody.

26. The method of claim 25, wherein IV dexamethasone is administered at a dose of 10 mg.

27. The method of claim 1, wherein oral dexamethasone is administered 1 day prior to the administration of the anti-EGFR/c-Met antibody.

28. The method of claim 27, wherein oral dexamethasone is administered at a total daily dose of 8 mg.

29. The method of claim 27, further comprising administration of IV dexamethasone on the first and second days of administering the anti-EGFR/c-Met antibody, wherein IV dexamethasone is administered at a dose between 10-20 mg.

30. The method of claim 1, further comprising administering a premedication with one or more of antihistamines, antipyretics, or glucocorticoids.

31. The method of claim 30, wherein the premedication comprises diphenhydramine.

32. The method of claim 31, wherein the diphenhydramine is administered at a dose of 25 to 50 mg.

33. The method of claim 30, wherein the premedication comprises acetaminophen.

34. The method of claim 33, wherein the acetaminophen is administered at a dose of 650 to 1,000 mg.


SEQUENCE LISTING (20 sequences)

SEQ ID NO: 1 - protein, 5 aa, Homo sapiens
TYGMH

SEQ ID NO: 2 - protein, 17 aa, Homo sapiens
VIWDDGSYKYYGDSVKG

SEQ ID NO: 3 - protein, 16 aa, Homo sapiens
DGITMVRGVMKDYFDY

SEQ ID NO: 4 - protein, 11 aa, Homo sapiens
RASQDISSALV

SEQ ID NO: 5 - protein, 7 aa, Homo sapiens
DASSLES

SEQ ID NO: 6 - protein, 9 aa, Homo sapiens
QQFNSYPLT

SEQ ID NO: 7 - protein, 5 aa, Homo sapiens
SYGIS

SEQ ID NO: 8 - protein, 17 aa, Homo sapiens
WISAYNGYTNYAQKLQG

SEQ ID NO: 9 - protein, 10 aa, Homo sapiens
DLRGTNYFDY

SEQ ID NO: 10 - protein, 11 aa, Homo sapiens
RASQGISNWLA

SEQ ID NO: 11 - protein, 7 aa, Homo sapiens
AASSLLS

SEQ ID NO: 12 - protein, 9 aa, Homo sapiens
QQANSFPIT

SEQ ID NO: 13 - protein, 125 aa, Homo sapiens [sequence omitted]

SEQ ID NO: 14 - protein, 107 aa, Homo sapiens [sequence omitted]

SEQ ID NO: 15 - protein, 119 aa, Homo sapiens
QVQLVQSGAEVKKPGASVKVSCETSGYTFTSYGISWVRQAPGHGLEWMGWISAYNGYTNYAQKLQGRVTMTTDTSTSTAYMELRSLRSDDTAVYYCARDLRGTNYFDYWGQGTLVTVSS

SEQ ID NO: 16 - protein, 107 aa, Homo sapiens
DIQMTQSPSSVSASVGDRVTITCRASQGISNWLAWFQHKPGKAPKLLIYAASSLLSGVPSRFSGSGSGTDFTLTISSLQPEDFATYYCQQANSFPITFGQGTRLEIK

SEQ ID NO: 17 - protein, 455 aa, Homo sapiens
QVQLVESGGGVVQPGRSLRLSCAASGFTFSTYGMHWVRQAPGKGLEWVAVIWDDGSYKYYGDSVKGRFTISRDNSKNTLYLQMNSLRAEDTAVYYCARDGITMVRGVMKDYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFLLYSKLTVDKSRWQQGNVFSCSVMHEALHNHYTQKSLSLSPGK

SEQ ID NO: 18 - protein, 214 aa, Homo sapiens
AIQLTQSPSSLSASVGDRVTITCRASQDISSALVWYQQKPGKAPKLLIYDASSLESGVPSRFSGSESGTDFTLTISSLQPEDFATYYCQQFNSYPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 19 - protein, 449 aa, Homo sapiens
QVQLVQSGAEVKKPGASVKVSCETSGYTFTSYGISWVRQAPGHGLEWMGWISAYNGYTNYAQKLQGRVTMTTDTSTSTAYMELRSLRSDDTAVYYCARDLRGTNYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSRLTVDKSRWQQGNVFSCSVMHEALHNHYTQKSLSLSPGK

SEQ ID NO: 20 - protein, 214 aa, Homo sapiens
DIQMTQSPSSVSASVGDRVTITCRASQGISNWLAWFQHKPGKAPKLLIYAASSLLSGVPSRFSGSGSGTDFTLTISSLQPEDFATYYCQQANSFPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC